SEQUENCE LISTING (450 sequences)

SEQ ID NO: 1 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 2 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 3 - protein, 4 aa, Artificial Sequence
  synthetic peptide
  PEPTIDE (1)..(1): any amino acid or non-natural amino acid excluding R, K, H, D, E, and A
XCRC

SEQ ID NO: 4 - protein, 5 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRC

SEQ ID NO: 5 - protein, 6 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRC

SEQ ID NO: 6 - protein, 5 aa, Artificial Sequence
  synthetic peptide
  PEPTIDE (1)..(1): any amino acid or non-natural amino acid excluding R, K, H, D, E, and A
  misc_feature (4)..(4): Xaa can be any naturally occurring amino acid
XCRXC

SEQ ID NO: 7 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  misc_feature (5)..(5): Xaa can be any naturally occurring amino acid
XXCRXC

SEQ ID NO: 8 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  misc_feature (6)..(6): Xaa can be any naturally occurring amino acid
XXXCRXC

SEQ ID NO: 9 - protein, 6 aa, Artificial Sequence
  synthetic peptide
  PEPTIDE (1)..(1): any amino acid or non-natural amino acid excluding R, K, H, D, E, and A
  misc_feature (4)..(5): Xaa can be any naturally occurring amino acid
XCRXXC

SEQ ID NO: 10 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  misc_feature (5)..(6): Xaa can be any naturally occurring amino acid
XXCRXXC

SEQ ID NO: 11 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  misc_feature (6)..(7): Xaa can be any naturally occurring amino acid
XXXCRXXC

SEQ ID NO: 12 - protein, 5 aa, Artificial Sequence
  synthetic peptide
  PEPTIDE (1)..(1): any amino acid or non-natural amino acid excluding R, K, H, D, E, and A
XCPYC

SEQ ID NO: 13 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYC

SEQ ID NO: 14 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYC

SEQ ID NO: 15 - protein, 5 aa, Artificial Sequence
  synthetic peptide
  PEPTIDE (1)..(1): any amino acid or non-natural amino acid excluding R, K, H, D, E, and A
XCGHC

SEQ ID NO: 16 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCGHC

SEQ ID NO: 17 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCGHC

SEQ ID NO: 18 - protein, 5 aa, Artificial Sequence
  synthetic peptide
  PEPTIDE (1)..(1): any amino acid or non-natural amino acid excluding R, K, H, D, E, and A
XCHGC

SEQ ID NO: 19 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCHGC

SEQ ID NO: 20 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCHGC

SEQ ID NO: 21 - protein, 5 aa, Artificial Sequence
  synthetic peptide
  PEPTIDE (1)..(1): any amino acid or non-natural amino acid excluding R, K, H, D, E, and A
XCRLC

SEQ ID NO: 22 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRLC

SEQ ID NO: 23 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRLC

SEQ ID NO: 24 - protein, 5 aa, Artificial Sequence
  synthetic peptide
  PEPTIDE (1)..(1): any amino acid or non-natural amino acid excluding R, K, H, D, E, and A
XCGFC

SEQ ID NO: 25 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCGFC

SEQ ID NO: 26 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCGFC

SEQ ID NO: 27 - protein, 5 aa, Artificial Sequence
  synthetic peptide
  PEPTIDE (1)..(1): any amino acid or non-natural amino acid excluding R, K, H, D, E, and A
XCHPC

SEQ ID NO: 28 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCHPC

SEQ ID NO: 29 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCHPC

SEQ ID NO: 30 - protein, 5 aa, Artificial Sequence
  synthetic peptide
  PEPTIDE (1)..(1): any amino acid or non-natural amino acid excluding R, K, H, D, E, and A
XCGPC

SEQ ID NO: 31 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCGPC

SEQ ID NO: 32 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCGPC

SEQ ID NO: 33 - protein, 3 aa, Artificial Sequence
  synthetic peptide
  PEPTIDE (1)..(1): any amino acid or non-natural amino acid excluding R, K, H, D, E, and A
XCC

SEQ ID NO: 34 - protein, 4 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCC

SEQ ID NO: 35 - protein, 5 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCC

SEQ ID NO: 36 - protein, 4 aa, Artificial Sequence
  synthetic peptide
  PEPTIDE (1)..(1): any amino acid or non-natural amino acid excluding R, K, H, D, E, and A
XCRC

SEQ ID NO: 37 - protein, 5 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRC

SEQ ID NO: 38 - protein, 6 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRC

SEQ ID NO: 39 - protein, 4 aa, Artificial Sequence
  synthetic peptide
  PEPTIDE (1)..(1): any amino acid or non-natural amino acid excluding R, K, H, D, E, and A
XCKC

SEQ ID NO: 40 - protein, 5 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKC

SEQ ID NO: 41 - protein, 6 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKC

SEQ ID NO: 42 - protein, 6 aa, Artificial Sequence
  synthetic peptide
  PEPTIDE (1)..(1): any amino acid or non-natural amino acid excluding R, K, H, D, E, and A
XCRPYC

SEQ ID NO: 43 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRPYC

SEQ ID NO: 44 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRPYC

SEQ ID NO: 45 - protein, 6 aa, Artificial Sequence
  synthetic peptide
  PEPTIDE (1)..(1): any amino acid or non-natural amino acid excluding R, K, H, D, E, and A
XCKPYC

SEQ ID NO: 46 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKPYC

SEQ ID NO: 47 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKPYC

SEQ ID NO: 48 - protein, 6 aa, Artificial Sequence
  synthetic peptide
  PEPTIDE (1)..(1): any amino acid or non-natural amino acid excluding R, K, H, D, E, and A
XCRGHC

SEQ ID NO: 49 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRGHC

SEQ ID NO: 50 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRGHC

SEQ ID NO: 51 - protein, 6 aa, Artificial Sequence
  synthetic peptide
  PEPTIDE (1)..(1): any amino acid or non-natural amino acid excluding R, K, H, D, E, and A
XCKGHC

SEQ ID NO: 52 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKGHC

SEQ ID NO: 53 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKGHC

SEQ ID NO: 54 - protein, 6 aa, Artificial Sequence
  synthetic peptide
  PEPTIDE (1)..(1): any amino acid or non-natural amino acid excluding R, K, H, D, E, and A
XCRHGC

SEQ ID NO: 55 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRHGC

SEQ ID NO: 56 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRHGC

SEQ ID NO: 57 - protein, 6 aa, Artificial Sequence
  synthetic peptide
  PEPTIDE (1)..(1): any amino acid or non-natural amino acid excluding R, K, H, D, E, and A
XCKHGC

SEQ ID NO: 58 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKHGC

SEQ ID NO: 59 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKHGC

SEQ ID NO: 60 - protein, 6 aa, Artificial Sequence
  synthetic peptide
  PEPTIDE (1)..(1): any amino acid or non-natural amino acid excluding R, K, H, D, E, and A
XCRRLC

SEQ ID NO: 61 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRRLC

SEQ ID NO: 62 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRRLC

SEQ ID NO: 63 - protein, 6 aa, Artificial Sequence
  synthetic peptide
  PEPTIDE (1)..(1): any amino acid or non-natural amino acid excluding R, K, H, D, E, and A
XCKRLC

SEQ ID NO: 64 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKRLC

SEQ ID NO: 65 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKRLC

SEQ ID NO: 66 - protein, 6 aa, Artificial Sequence
  synthetic peptide
  PEPTIDE (1)..(1): any amino acid or non-natural amino acid excluding R, K, H, D, E, and A
XCRGFC

SEQ ID NO: 67 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRGFC

SEQ ID NO: 68 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRGFC

SEQ ID NO: 69 - protein, 6 aa, Artificial Sequence
  synthetic peptide
  PEPTIDE (1)..(1): any amino acid or non-natural amino acid excluding R, K, H, D, E, and A
XCKGFC

SEQ ID NO: 70 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKGFC

SEQ ID NO: 71 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKGFC

SEQ ID NO: 72 - protein, 6 aa, Artificial Sequence
  synthetic peptide
  PEPTIDE (1)..(1): any amino acid or non-natural amino acid excluding R, K, H, D, E, and A
XCRHPC

SEQ ID NO: 73 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRHPC

SEQ ID NO: 74 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRHPC

SEQ ID NO: 75 - protein, 6 aa, Artificial Sequence
  synthetic peptide
  PEPTIDE (1)..(1): any amino acid or non-natural amino acid excluding R, K, H, D, E, and A
XCKHPC

SEQ ID NO: 76 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKHPC

SEQ ID NO: 77 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKHPC

SEQ ID NO: 78 - protein, 6 aa, Artificial Sequence
  synthetic peptide
  PEPTIDE (1)..(1): any amino acid or non-natural amino acid excluding R, K, H, D, E, and A
XCRGPC

SEQ ID NO: 79 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCRGPC

SEQ ID NO: 80 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCRGPC

SEQ ID NO: 81 - protein, 6 aa, Artificial Sequence
  synthetic peptide
  PEPTIDE (1)..(1): any amino acid or non-natural amino acid excluding R, K, H, D, E, and A
XCKGPC

SEQ ID NO: 82 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCKGPC

SEQ ID NO: 83 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCKGPC

SEQ ID NO: 84 - protein, 20 aa, Artificial Sequence
  synthetic peptide
  PEPTIDE (1)..(1): any amino acid or non-natural amino acid excluding R, K, H, D, E, and A
XCPYCGWYRSPFSRVVHLYR

SEQ ID NO: 85 - protein, 21 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYCGWYRSPFSRVVHLYR

SEQ ID NO: 86 - protein, 22 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYCGWYRSPFSRVVHLYR

SEQ ID NO: 87 - protein, 17 aa, Artificial Sequence
  synthetic peptide
  PEPTIDE (1)..(1): any amino acid or non-natural amino acid excluding R, K, H, D, E, and A
XCPYCGWYRSPFSRVVK

SEQ ID NO: 88 - protein, 18 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYCGWYRSPFSRVVK

SEQ ID NO: 89 - protein, 19 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYCGWYRSPFSRVVK

SEQ ID NO: 90 - protein, 20 aa, Artificial Sequence
  synthetic peptide
  PEPTIDE (1)..(1): any amino acid or non-natural amino acid excluding R, K, H, D, E, and A
XCPYCVRYFLRVPSWKITLF

SEQ ID NO: 91 - protein, 21 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYCVRYFLRVPSWKITLF

SEQ ID NO: 92 - protein, 22 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYCVRYFLRVPSWKITLF

SEQ ID NO: 93 - protein, 20 aa, Artificial Sequence
  synthetic peptide
  PEPTIDE (1)..(1): any amino acid or non-natural amino acid excluding R, K, H, D, E, and A
XCPYCSLQPLALEGSLQKRG

SEQ ID NO: 94 - protein, 21 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYCSLQPLALEGSLQKRG

SEQ ID NO: 95 - protein, 22 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYCSLQPLALEGSLQKRG

SEQ ID NO: 96 - protein, 4 aa, Artificial Sequence
  synthetic peptide
  PEPTIDE (1)..(1): any amino acid or non-natural amino acid excluding R, K, H, D, E, and A
  PEPTIDE (2)..(2): C, S, or T
  misc_feature (3)..(3): Xaa can be any naturally occurring amino acid
XXXC

SEQ ID NO: 97 - protein, 5 aa, Artificial Sequence
  synthetic peptide
  PEPTIDE (1)..(1): any amino acid or non-natural amino acid excluding R, K, H, D, E, and A
  PEPTIDE (2)..(2): C, S, or T
  misc_feature (3)..(4): Xaa can be any naturally occurring amino acid
XXXXC

SEQ ID NO: 98 - protein, 6 aa, Artificial Sequence
  synthetic peptide
  PEPTIDE (1)..(1): any amino acid or non-natural amino acid excluding R, K, H, D, E, and A
  PEPTIDE (2)..(2): C, S, or T
  misc_feature (3)..(5): Xaa can be any naturally occurring amino acid
XXXXXC

SEQ ID NO: 99 - protein, 7 aa, Artificial Sequence
  synthetic peptide
  PEPTIDE (1)..(1): any amino acid or non-natural amino acid excluding R, K, H, D, E, and A
  PEPTIDE (2)..(2): C, S, or T
  misc_feature (3)..(6): Xaa can be any naturally occurring amino acid
XXXXXXC

SEQ ID NO: 100 - protein, 4 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (3)..(3): C, S, or T
XXXC

SEQ ID NO: 101 - protein, 5 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (3)..(3): C, S, or T
  misc_feature (4)..(4): Xaa can be any naturally occurring amino acid
XXXXC

SEQ ID NO: 102 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (3)..(3): C, S, or T
  misc_feature (4)..(5): Xaa can be any naturally occurring amino acid
XXXXXC

SEQ ID NO: 103 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (3)..(3): C, S, or T
  misc_feature (4)..(6): Xaa can be any naturally occurring amino acid
XXXXXXC

SEQ ID NO: 104 - protein, 8 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (3)..(3): C, S, or T
  misc_feature (4)..(7): Xaa can be any naturally occurring amino acid
XXXXXXXC

SEQ ID NO: 105 - protein, 5 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (4)..(4): C, S, or T
XXXXC

SEQ ID NO: 106 - protein, 6 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (4)..(4): C, S, or T
  misc_feature (5)..(5): Xaa can be any naturally occurring amino acid
XXXXXC

SEQ ID NO: 107 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (4)..(4): C, S, or T
  misc_feature (5)..(6): Xaa can be any naturally occurring amino acid
XXXXXXC

SEQ ID NO: 108 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (4)..(4): C, S, or T
  misc_feature (5)..(7): Xaa can be any naturally occurring amino acid
XXXXXXXC

SEQ ID NO: 109 - protein, 9 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (4)..(4): C, S, or T
  misc_feature (5)..(8): Xaa can be any naturally occurring amino acid
XXXXXXXXC

SEQ ID NO: 110 - protein, 4 aa, Artificial Sequence
  synthetic peptide
  PEPTIDE (1)..(1): any amino acid or non-natural amino acid excluding R, K, H, D, E, and A
  misc_feature (3)..(3): Xaa can be any naturally occurring amino acid
  PEPTIDE (4)..(4): C, S, or T
XCXX

SEQ ID NO: 111 - protein, 5 aa, Artificial Sequence
  synthetic peptide
  PEPTIDE (1)..(1): any amino acid or non-natural amino acid excluding R, K, H, D, E, and A
  misc_feature (3)..(4): Xaa can be any naturally occurring amino acid
  PEPTIDE (5)..(5): C, S, or T
XCXXX

SEQ ID NO: 112 - protein, 6 aa, Artificial Sequence
  synthetic peptide
  PEPTIDE (1)..(1): any amino acid or non-natural amino acid excluding R, K, H, D, E, and A
  misc_feature (3)..(5): Xaa can be any naturally occurring amino acid
  PEPTIDE (6)..(6): C, S, or T
XCXXXX

SEQ ID NO: 113 - protein, 7 aa, Artificial Sequence
  synthetic peptide
  PEPTIDE (1)..(1): any amino acid or non-natural amino acid excluding R, K, H, D, E, and A
  misc_feature (3)..(6): Xaa can be any naturally occurring amino acid
  PEPTIDE (7)..(7): C, S, or T
XCXXXXX

SEQ ID NO: 114 - protein, 4 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (4)..(4): C, S, or T
XXCX

SEQ ID NO: 115 - protein, 5 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  misc_feature (4)..(4): Xaa can be any naturally occurring amino acid
  PEPTIDE (5)..(5): C, S, or T
XXCXX

SEQ ID NO: 116 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  misc_feature (4)..(5): Xaa can be any naturally occurring amino acid
  PEPTIDE (6)..(6): C, S, or T
XXCXXX

SEQ ID NO: 117 - protein, 7 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  misc_feature (4)..(6): Xaa can be any naturally occurring amino acid
  PEPTIDE (7)..(7): C, S, or T
XXCXXXX

SEQ ID NO: 118 - protein, 8 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  misc_feature (4)..(7): Xaa can be any naturally occurring amino acid
  PEPTIDE (8)..(8): C, S, or T
XXCXXXXX

SEQ ID NO: 119 - protein, 5 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (5)..(5): C, S, or T
XXXCX

SEQ ID NO: 120 - protein, 6 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  misc_feature (5)..(5): Xaa can be any naturally occurring amino acid
  PEPTIDE (6)..(6): C, S, or T
XXXCXX

SEQ ID NO: 121 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  misc_feature (5)..(6): Xaa can be any naturally occurring amino acid
  PEPTIDE (7)..(7): C, S, or T
XXXCXXX

SEQ ID NO: 122 - protein, 8 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  misc_feature (5)..(7): Xaa can be any naturally occurring amino acid
  PEPTIDE (8)..(8): C, S, or T
XXXCXXXX

SEQ ID NO: 123 - protein, 9 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  misc_feature (5)..(8): Xaa can be any naturally occurring amino acid
  PEPTIDE (9)..(9): C, S, or T
XXXCXXXXX

SEQ ID NO: 124 - protein, 22 aa, Artificial Sequence
  synthetic peptide
  PEPTIDE (1)..(1): any amino acid or non-natural amino acid excluding R, K, H, D, E, and A
XCPYCVRYFLRVPSWKITLFKK

SEQ ID NO: 125 - protein, 23 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYCVRYFLRVPSWKITLFKK

SEQ ID NO: 126 - protein, 24 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYCVRYFLRVPSWKITLFKK

SEQ ID NO: 127 - protein, 7 aa, Artificial Sequence
  synthetic peptide
  PEPTIDE (1)..(1): F, W, Y, H, or T
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, Y, H, or T
XXXXXXX

SEQ ID NO: 128 - protein, 7 aa, Artificial Sequence
  synthetic peptide
  PEPTIDE (1)..(1): F, W, Y, or H
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, Y, or H
XXXXXXX

SEQ ID NO: 129 - protein, 7 aa, Artificial Sequence
  synthetic peptide
  PEPTIDE (1)..(1): F, W, Y, or T
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, Y, or T
XXXXXXX

SEQ ID NO: 130 - protein, 7 aa, Artificial Sequence
  synthetic peptide
  PEPTIDE (1)..(1): F, W, or Y
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, or Y
XXXXXXX

SEQ ID NO: 131 - protein, 7 aa, Artificial Sequence
  synthetic peptide
  PEPTIDE (1)..(1): F, W, Y, or H
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, Y, or H
XXXXXXX

SEQ ID NO: 132 - protein, 7 aa, Artificial Sequence
  synthetic peptide
  PEPTIDE (1)..(1): F, W, Y, H, or T
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, Y, H, or T
XXXXXXX

SEQ ID NO: 133 - protein, 7 aa, Artificial Sequence
  synthetic peptide
  PEPTIDE (1)..(1): F, W, or Y
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, or Y
XXXXXXX

SEQ ID NO: 134 - protein, 7 aa, Artificial Sequence
  synthetic peptide
  PEPTIDE (1)..(1): F or W
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, T, or H
XXXXXXX

SEQ ID NO: 135 - protein, 7 aa, Artificial Sequence
  synthetic peptide
  PEPTIDE (1)..(1): F, W, T, or H
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F or W
XXXXXXX

SEQ ID NO: 136 - protein, 5 aa, Artificial Sequence
  synthetic peptide
  PEPTIDE (1)..(1): any amino acid or non-natural amino acid excluding R, K, H, D, E, and A
  PEPTIDE (2)..(2): C, S, or T
XXPYC

SEQ ID NO: 137 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (3)..(3): C, S, or T
XXXPYC

SEQ ID NO: 138 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (4)..(4): C, S, or T
XXXXPYC

SEQ ID NO: 139 - protein, 5 aa, Artificial Sequence
  synthetic peptide
  PEPTIDE (1)..(1): any amino acid or non-natural amino acid excluding R, K, H, D, E, and A
  PEPTIDE (5)..(5): C, S, or T
XCPYX

SEQ ID NO: 140 - protein, 6 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
  PEPTIDE (6)..(6): C, S, or T
XXCPYX

SEQ ID NO: 141 - protein, 7 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
  PEPTIDE (7)..(7): C, S, or T
XXXCPYX

SEQ ID NO: 142 - protein, 5 aa, Artificial Sequence
  synthetic peptide
  misc_feature (1)..(1): Xaa can be any naturally occurring amino acid
XCPYC

SEQ ID NO: 143 - protein, 6 aa, Artificial Sequence
  synthetic peptide
  misc_feature (1)..(2): Xaa can be any naturally occurring amino acid
XXCPYC

SEQ ID NO: 144 - protein, 7 aa, Artificial Sequence
  synthetic peptide
  misc_feature (1)..(3): Xaa can be any naturally occurring amino acid
XXXCPYC

SEQ ID NO: 145 - protein, 5 aa, Artificial Sequence
  synthetic peptide
  misc_feature (1)..(1): Xaa can be any naturally occurring amino acid
XSPYC

SEQ ID NO: 146 - protein, 6 aa, Artificial Sequence
  synthetic peptide
  misc_feature (1)..(2): Xaa can be any naturally occurring amino acid
XXSPYC

SEQ ID NO: 147 - protein, 7 aa, Artificial Sequence
  synthetic peptide
  misc_feature (1)..(3): Xaa can be any naturally occurring amino acid
XXXSPYC

SEQ ID NO: 148 - protein, 5 aa, Artificial Sequence
  synthetic peptide
  misc_feature (1)..(1): Xaa can be any naturally occurring amino acid
XTPYC

SEQ ID NO: 149 - protein, 6 aa, Artificial Sequence
  synthetic peptide
  misc_feature (1)..(2): Xaa can be any naturally occurring amino acid
XXTPYC

SEQ ID NO: 150 - protein, 7 aa, Artificial Sequence
  synthetic peptide
  misc_feature (1)..(3): Xaa can be any naturally occurring amino acid
XXXTPYC

SEQ ID NO: 151 - protein, 5 aa, Artificial Sequence
  synthetic peptide
  misc_feature (1)..(1): Xaa can be any naturally occurring amino acid
XCPYC

SEQ ID NO: 152 - protein, 6 aa, Artificial Sequence
  synthetic peptide
  misc_feature (1)..(2): Xaa can be any naturally occurring amino acid
XXCPYC

SEQ ID NO: 153 - protein, 7 aa, Artificial Sequence
  synthetic peptide
  misc_feature (1)..(3): Xaa can be any naturally occurring amino acid
XXXCPYC

SEQ ID NO: 154 - protein, 5 aa, Artificial Sequence
  synthetic peptide
  misc_feature (1)..(1): Xaa can be any naturally occurring amino acid
XCPYS

SEQ ID NO: 155 - protein, 6 aa, Artificial Sequence
  synthetic peptide
  misc_feature (1)..(2): Xaa can be any naturally occurring amino acid
XXCPYS

SEQ ID NO: 156 - protein, 7 aa, Artificial Sequence
  synthetic peptide
  misc_feature (1)..(3): Xaa can be any naturally occurring amino acid
XXXCPYS

SEQ ID NO: 157 - protein, 5 aa, Artificial Sequence
  synthetic peptide
  misc_feature (1)..(1): Xaa can be any naturally occurring amino acid
XCPYT

SEQ ID NO: 158 - protein, 6 aa, Artificial Sequence
  synthetic peptide
  misc_feature (1)..(2): Xaa can be any naturally occurring amino acid
XXCPYT

SEQ ID NO: 159 - protein, 7 aa, Artificial Sequence
  synthetic peptide
  misc_feature (1)..(3): Xaa can be any naturally occurring amino acid
XXXCPYT

SEQ ID NO: 160 - protein, 5 aa, Artificial Sequence
  synthetic peptide
  misc_feature (1)..(1): Xaa can be any naturally occurring amino acid
  PEPTIDE (2)..(2): C, S, or T
XXHGC

SEQ ID NO: 161 - protein, 6 aa, Artificial Sequence
  synthetic peptide
  misc_feature (1)..(2): Xaa can be any naturally occurring amino acid
  PEPTIDE (3)..(3): C, S, or T
XXXHGC

SEQ ID NO: 162 - protein, 7 aa, Artificial Sequence
  synthetic peptide
  misc_feature (1)..(3): Xaa can be any naturally occurring amino acid
  PEPTIDE (4)..(4): C, S, or T
XXXXHGC

SEQ ID NO: 163 - protein, 5 aa, Artificial Sequence
  synthetic peptide
  misc_feature (1)..(1): Xaa can be any naturally occurring amino acid
  PEPTIDE (5)..(5): C, S, or T
XCHGX

SEQ ID NO: 164 - protein, 6 aa, Artificial Sequence
  synthetic peptide
  misc_feature (1)..(2): Xaa can be any naturally occurring amino acid
  PEPTIDE (6)..(6): C, S, or T
XXCHGX

SEQ ID NO: 165 - protein, 7 aa, Artificial Sequence
  synthetic peptide
  misc_feature (1)..(3): Xaa can be any naturally occurring amino acid
  PEPTIDE (7)..(7): C, S, or T
XXXCHGX

SEQ ID NO: 166 - protein, 5 aa, Artificial Sequence
  synthetic peptide
  misc_feature (1)..(1): Xaa can be any naturally occurring amino acid
XCHGC

SEQ ID NO: 167 - protein, 6 aa, Artificial Sequence
  synthetic peptide
  misc_feature (1)..(2): Xaa can be any naturally occurring amino acid
XXCHGC

SEQ ID NO: 168 - protein, 7 aa, Artificial Sequence
  synthetic peptide
  misc_feature (1)..(3): Xaa can be any naturally occurring amino acid
XXXCHGC

SEQ ID NO: 169 - protein, 5 aa, Artificial Sequence
  synthetic peptide
  misc_feature (1)..(1): Xaa can be any naturally occurring amino acid
XSHGC

SEQ ID NO: 170 - protein, 6 aa, Artificial Sequence
  synthetic peptide
  misc_feature (1)..(2): Xaa can be any naturally occurring amino acid
XXSHGC

SEQ ID NO: 171 - protein, 7 aa, Artificial Sequence
  synthetic peptide
  misc_feature (1)..(3): Xaa can be any naturally occurring amino acid
XXXSHGC

SEQ ID NO: 172 - protein, 5 aa, Artificial Sequence
  synthetic peptide
  misc_feature (1)..(1): Xaa can be any naturally occurring amino acid
XTHGC

SEQ ID NO: 173 - protein, 6 aa, Artificial Sequence
  synthetic peptide
  misc_feature (1)..(2): Xaa can be any naturally occurring amino acid
XXTHGC

SEQ ID NO: 174 - protein, 7 aa, Artificial Sequence
  synthetic peptide
  misc_feature (1)..(3): Xaa can be any naturally occurring amino acid
XXXTHGC

SEQ ID NO: 175 - protein, 5 aa, Artificial Sequence
  synthetic peptide
  misc_feature (1)..(1): Xaa can be any naturally occurring amino acid
XCHGC

SEQ ID NO: 176 - protein, 6 aa, Artificial Sequence
  synthetic peptide
  misc_feature (1)..(2): Xaa can be any naturally occurring amino acid
XXCHGC

SEQ ID NO: 177 - protein, 7 aa, Artificial Sequence
  synthetic peptide
  misc_feature (1)..(3): Xaa can be any naturally occurring amino acid
XXXCHGC

SEQ ID NO: 178 - protein, 5 aa, Artificial Sequence
  synthetic peptide
  misc_feature (1)..(1): Xaa can be any naturally occurring amino acid
XCHGS

SEQ ID NO: 179 - protein, 6 aa, Artificial Sequence
  synthetic peptide
  misc_feature (1)..(2): Xaa can be any naturally occurring amino acid
XXCHGS

SEQ ID NO: 180 - protein, 7 aa, Artificial Sequence
  synthetic peptide
  misc_feature (1)..(3): Xaa can be any naturally occurring amino acid
XXXCHGS

SEQ ID NO: 181 - protein, 5 aa, Artificial Sequence
  synthetic peptide
  misc_feature (1)..(1): Xaa can be any naturally occurring amino acid
XCHGT

SEQ ID NO: 182 - protein, 6 aa, Artificial Sequence
  synthetic peptide
  misc_feature (1)..(2): Xaa can be any naturally occurring amino acid
XXCHGT

SEQ ID NO: 183 - protein, 7 aa, Artificial Sequence
  synthetic peptide
  misc_feature (1)..(3): Xaa can be any naturally occurring amino acid
XXXCHGT

SEQ ID NO: 184 - protein, 5 aa, Artificial Sequence
  synthetic peptide
  misc_feature (1)..(1): Xaa can be any naturally occurring amino acid
  PEPTIDE (2)..(2): C, S, or T
XXGPC

SEQ ID NO: 185 - protein, 6 aa, Artificial Sequence
  synthetic peptide
  misc_feature (1)..(2): Xaa can be any naturally occurring amino acid
  PEPTIDE (3)..(3): C, S, or T
XXXGPC

SEQ ID NO: 186 - protein, 7 aa, Artificial Sequence
  synthetic peptide
  misc_feature (1)..(3): Xaa can be any naturally occurring amino acid
  PEPTIDE (4)..(4): C, S, or T
XXXXGPC

SEQ ID NO: 187 - protein, 5 aa, Artificial Sequence
  synthetic peptide
  misc_feature (1)..(1): Xaa can be any naturally occurring amino acid
  PEPTIDE (5)..(5): C, S, or T
XCGPX

SEQ ID NO: 188 - protein, 6 aa, Artificial Sequence
  synthetic peptide
  misc_feature (1)..(2): Xaa can be any naturally occurring amino acid
  PEPTIDE (6)..(6): C, S, or T
XXCGPX

SEQ ID NO: 189 - protein, 7 aa, Artificial Sequence
  synthetic peptide
  misc_feature (1)..(3): Xaa can be any naturally occurring amino acid
  PEPTIDE (7)..(7): C, S, or T
XXXCGPX

SEQ ID NO: 190 - protein, 5 aa, Artificial Sequence
  synthetic peptide
  misc_feature (1)..(1): Xaa can be any naturally occurring amino acid
XCGPC

SEQ ID NO: 191 - protein, 6 aa, Artificial Sequence
  synthetic peptide
  misc_feature (1)..(2): Xaa can be any naturally occurring amino acid
XXCGPC

SEQ ID NO: 192 - protein, 7 aa, Artificial Sequence
  synthetic peptide
  misc_feature (1)..(3): Xaa can be any naturally occurring amino acid
XXXCGPC

SEQ ID NO: 193 - protein, 5 aa, Artificial Sequence
  synthetic peptide
  misc_feature (1)..(1): Xaa can be any naturally occurring amino acid
XSGPC

SEQ ID NO: 194 - protein, 6 aa, Artificial Sequence
  synthetic peptide
  misc_feature (1)..(2): Xaa can be any naturally occurring amino acid
XXSGPC

SEQ ID NO: 195 - protein, 7 aa, Artificial Sequence
  synthetic peptide
  misc_feature (1)..(3): Xaa can be any naturally occurring amino acid
XXXSGPC

SEQ ID NO: 196 - protein, 5 aa, Artificial Sequence
  synthetic peptide
  misc_feature (1)..(1): Xaa can be any naturally occurring amino acid
XTGPC

SEQ ID NO: 197 - protein, 6 aa, Artificial Sequence
  synthetic peptide
  misc_feature (1)..(2): Xaa can be any naturally occurring amino acid
XXTGPC

SEQ ID NO: 198 - protein, 7 aa, Artificial Sequence
  synthetic peptide
  misc_feature (1)..(3): Xaa can be any naturally occurring amino acid
XXXTGPC

SEQ ID NO: 199 - protein, 5 aa, Artificial Sequence
  synthetic peptide
  misc_feature (1)..(1): Xaa can be any naturally occurring amino acid
XCGPC

SEQ ID NO: 200 - protein, 6 aa, Artificial Sequence
  synthetic peptide
  misc_feature (1)..(2): Xaa can be any naturally occurring amino acid
XXCGPC

SEQ ID NO: 201 - protein, 7 aa, Artificial Sequence
  synthetic peptide
  misc_feature (1)..(3): Xaa can be any naturally occurring amino acid
XXXCGPC

SEQ ID NO: 202 - protein, 5 aa, Artificial Sequence
  synthetic peptide
  misc_feature (1)..(1): Xaa can be any naturally occurring amino acid
XCGPS

SEQ ID NO: 203 - protein, 6 aa, Artificial Sequence
  synthetic peptide
  misc_feature (1)..(2): Xaa can be any naturally occurring amino acid
XXCGPS

SEQ ID NO: 204 - protein, 7 aa, Artificial Sequence
  synthetic peptide
  misc_feature (1)..(3): Xaa can be any naturally occurring amino acid
XXXCGPS

SEQ ID NO: 205 - protein, 5 aa, Artificial Sequence
  synthetic peptide
  misc_feature (1)..(1): Xaa can be any naturally occurring amino acid
XCGPT

SEQ ID NO: 206 - protein, 6 aa, Artificial Sequence
  synthetic peptide
  misc_feature (1)..(2): Xaa can be any naturally occurring amino acid
XXCGPT

SEQ ID NO: 207 - protein, 7 aa, Artificial Sequence
  synthetic peptide
  misc_feature (1)..(3): Xaa can be any naturally occurring amino acid
XXXCGPT

SEQ ID NO: 208 - protein, 5 aa, Artificial Sequence
  synthetic peptide
  misc_feature (1)..(1): Xaa can be any naturally occurring amino acid
  PEPTIDE (2)..(2): C, S, or T
XXGHC

SEQ ID NO: 209 - protein, 6 aa, Artificial Sequence
  synthetic peptide
  misc_feature (1)..(2): Xaa can be any naturally occurring amino acid
  PEPTIDE (3)..(3): C, S, or T
XXXGHC

SEQ ID NO: 210 - protein, 7 aa, Artificial Sequence
  synthetic peptide
  misc_feature (1)..(3): Xaa can be any naturally occurring amino acid
  PEPTIDE (4)..(4): C, S, or T
XXXXGHC

SEQ ID NO: 211 - protein, 5 aa, Artificial Sequence
  synthetic peptide
  misc_feature (1)..(1): Xaa can be any naturally occurring amino acid
  PEPTIDE (5)..(5): C, S, or T
XCGHX

SEQ ID NO: 212 - protein, 6 aa, Artificial Sequence
  synthetic peptide
  misc_feature (1)..(2): Xaa can be any naturally occurring amino acid
  PEPTIDE (6)..(6): C, S, or T
XXCGHX

SEQ ID NO: 213 - protein, 7 aa, Artificial Sequence
  synthetic peptide
  misc_feature (1)..(3): Xaa can be any naturally occurring amino acid
  PEPTIDE (7)..(7): C, S, or T
XXXCGHX

SEQ ID NO: 214 - protein, 5 aa, Artificial Sequence
  synthetic peptide
  misc_feature (1)..(1): Xaa can be any naturally occurring amino acid
XCGHC

SEQ ID NO: 215 - protein, 6 aa, Artificial Sequence
  synthetic peptide
  misc_feature (1)..(2): Xaa can be any naturally occurring amino acid
XXCGHC

SEQ ID NO: 216 - protein, 7 aa, Artificial Sequence
  synthetic peptide
  misc_feature (1)..(3): Xaa can be any naturally occurring amino acid
XXXCGHC

SEQ ID NO: 217 - protein, 5 aa, Artificial Sequence
  synthetic peptide
  misc_feature (1)..(1): Xaa can be any naturally occurring amino acid
XSGHC

SEQ ID NO: 218 - protein, 6 aa, Artificial Sequence
  synthetic peptide
  misc_feature (1)..(2): Xaa can be any naturally occurring amino acid
XXSGHC

SEQ ID NO: 219 - protein, 7 aa, Artificial Sequence
  synthetic peptide
  misc_feature (1)..(3): Xaa can be any naturally occurring amino acid
XXXSGHC

SEQ ID NO: 220 - protein, 5 aa, Artificial Sequence
  synthetic peptide
  misc_feature (1)..(1): Xaa can be any naturally occurring amino acid
XTGHC

SEQ ID NO: 221 - protein, 6 aa, Artificial Sequence
  synthetic peptide
  misc_feature (1)..(2): Xaa can be any naturally occurring amino acid
XXTGHC

SEQ ID NO: 222 - protein, 7 aa, Artificial Sequence
  synthetic peptide
  misc_feature (1)..(3): Xaa can be any naturally occurring amino acid
XXXTGHC

SEQ ID NO: 223 - protein, 5 aa, Artificial Sequence
  synthetic peptide
  misc_feature (1)..(1): Xaa can be any naturally occurring amino acid
XCGHC

SEQ ID NO: 224 - protein, 6 aa, Artificial Sequence
  synthetic peptide
  misc_feature (1)..(2): Xaa can be any naturally occurring amino acid
XXCGHC

SEQ ID NO: 225 - protein, 7 aa, Artificial Sequence
  synthetic peptide
  misc_feature (1)..(3): Xaa can be any naturally occurring amino acid
XXXCGHC

SEQ ID NO: 226 - protein, 5 aa, Artificial Sequence
  synthetic peptide
  misc_feature (1)..(1): Xaa can be any naturally occurring amino acid
XCGHS

SEQ ID NO: 227 - protein, 6 aa, Artificial Sequence
  synthetic peptide
  misc_feature (1)..(2): Xaa can be any naturally occurring amino acid
XXCGHS

SEQ ID NO: 228 - protein, 7 aa, Artificial Sequence
  synthetic peptide
  misc_feature (1)..(3): Xaa can be any naturally occurring amino acid
XXXCGHS

SEQ ID NO: 229 - protein, 5 aa, Artificial Sequence
  synthetic peptide
  misc_feature (1)..(1): Xaa can be any naturally occurring amino acid
XCGHT

SEQ ID NO: 230 - protein, 6 aa, Artificial Sequence
  synthetic peptide
  misc_feature (1)..(2): Xaa can be any naturally occurring amino acid
XXCGHT

SEQ ID NO: 231 - protein, 7 aa, Artificial Sequence
  synthetic peptide
  misc_feature (1)..(3): Xaa can be any naturally occurring amino acid
XXXCGHT

SEQ ID NO: 232 - protein, 5 aa, Artificial Sequence
  synthetic peptide
  misc_feature (1)..(1): Xaa can be any naturally occurring amino acid
  PEPTIDE (2)..(2): C, S, or T
XXGFC

SEQ ID NO: 233 - protein, 6 aa, Artificial Sequence
  synthetic peptide
  misc_feature (1)..(2): Xaa can be any naturally occurring amino acid
  PEPTIDE (3)..(3): C, S, or T
XXXGFC

SEQ ID NO: 234 - protein, 7 aa, Artificial Sequence
  synthetic peptide
  misc_feature (1)..(3): Xaa can be any naturally occurring amino acid
  PEPTIDE (4)..(4): C, S, or T
XXXXGFC

SEQ ID NO: 235 - protein, 5 aa, Artificial Sequence
  synthetic peptide
  misc_feature (1)..(1): Xaa can be any naturally occurring amino acid
  PEPTIDE (5)..(5): C, S, or T
XCGFX

SEQ ID NO: 236 - protein, 6 aa, Artificial Sequence
  synthetic peptide
  misc_feature (1)..(2): Xaa can be any naturally occurring amino acid
  PEPTIDE (6)..(6): C, S, or T
XXCGFX

SEQ ID NO: 237 - protein, 7 aa, Artificial Sequence
  synthetic peptide
  misc_feature (1)..(3): Xaa can be any naturally occurring amino acid
  PEPTIDE (7)..(7): C, S, or T
XXXCGFX

SEQ ID NO: 238 - protein, 5 aa, Artificial Sequence
  synthetic peptide
  misc_feature (1)..(1): Xaa can be any naturally occurring amino acid
XCGFC

SEQ ID NO: 239 - protein, 6 aa, Artificial Sequence
  synthetic peptide
  misc_feature (1)..(2): Xaa can be any naturally occurring amino acid
XXCGFC

SEQ ID NO: 240 - protein, 7 aa, Artificial Sequence
  synthetic peptide
  misc_feature (1)..(3): Xaa can be any naturally occurring amino acid
XXXCGFC

SEQ ID NO: 241 - protein, 5 aa, Artificial Sequence
  synthetic peptide
  misc_feature (1)..(1): Xaa can be any naturally occurring amino acid
XSGFC

SEQ ID NO: 242 - protein, 6 aa, Artificial Sequence
  synthetic peptide
  misc_feature (1)..(2): Xaa can be any naturally occurring amino acid
XXSGFC

SEQ ID NO: 243 - protein, 7 aa, Artificial Sequence
  synthetic peptide
  misc_feature (1)..(3): Xaa can be any naturally occurring amino acid
XXXSGFC

SEQ ID NO: 244 - protein, 5 aa, Artificial Sequence
  synthetic peptide
  misc_feature (1)..(1): Xaa can be any naturally occurring amino acid
XTGFC

SEQ ID NO: 245 - protein, 6 aa, Artificial Sequence
  synthetic peptide
  misc_feature (1)..(2): Xaa can be any naturally occurring amino acid
XXTGFC

SEQ ID NO: 246 - protein, 7 aa, Artificial Sequence
  synthetic peptide
  misc_feature (1)..(3): Xaa can be any naturally occurring amino acid
XXXTGFC

SEQ ID NO: 247 - protein, 5 aa, Artificial Sequence
  synthetic peptide
  misc_feature (1)..(1): Xaa can be any naturally occurring amino acid
XCGFC

SEQ ID NO: 248 - protein, 6 aa, Artificial Sequence
  synthetic peptide
  misc_feature (1)..(2): Xaa can be any naturally occurring amino acid
XXCGFC

SEQ ID NO: 249 - protein, 7 aa, Artificial Sequence
  synthetic peptide
  misc_feature (1)..(3): Xaa can be any naturally occurring amino acid
XXXCGFC

SEQ ID NO: 250 - protein, 5 aa, Artificial Sequence
  synthetic peptide
  misc_feature (1)..(1): Xaa can be any naturally occurring amino acid
XCGFS

SEQ ID NO: 251 - protein, 6 aa, Artificial Sequence
  synthetic peptide
  misc_feature (1)..(2): Xaa can be any naturally occurring amino acid
XXCGFS

SEQ ID NO: 252 - protein, 7 aa, Artificial Sequence
  synthetic peptide
  misc_feature (1)..(3): Xaa can be any naturally occurring amino acid
XXXCGFS

SEQ ID NO: 253 - protein, 5 aa, Artificial Sequence
  synthetic peptide
  misc_feature (1)..(1): Xaa can be any naturally occurring amino acid
XCGFT

SEQ ID NO: 254 - protein, 6 aa, Artificial Sequence
  synthetic peptide
  misc_feature (1)..(2): Xaa can be any naturally occurring amino acid
XXCGFT

SEQ ID NO: 255 - protein, 7 aa, Artificial Sequence
  synthetic peptide
  misc_feature (1)..(3): Xaa can be any naturally occurring amino acid
XXXCGFT

SEQ ID NO: 256 - protein, 5 aa, Artificial Sequence
  synthetic peptide
  misc_feature (1)..(1): Xaa can be any naturally occurring amino acid
  PEPTIDE (2)..(2): C, S, or T
XXRLC

SEQ ID NO: 257 - protein, 6 aa, Artificial Sequence
  synthetic peptide
  misc_feature (1)..(2): Xaa can be any naturally occurring amino acid
  PEPTIDE (3)..(3): C, S, or T
XXXRLC

SEQ ID NO: 258 - protein, 7 aa, Artificial Sequence
  synthetic peptide
  misc_feature (1)..(3): Xaa can be any naturally occurring amino acid
  PEPTIDE (4)..(4): C, S, or T
XXXXRLC

SEQ ID NO: 259 - protein, 5 aa, Artificial Sequence
  synthetic peptide
  misc_feature (1)..(1): Xaa can be any naturally occurring amino acid
  PEPTIDE (5)..(5): C, S, or T
XCRLX

SEQ ID NO: 260 - protein, 6 aa, Artificial Sequence
  synthetic peptide
  misc_feature (1)..(2): Xaa can be any naturally occurring amino acid
  PEPTIDE (6)..(6): C, S, or T
XXCRLX

SEQ ID NO: 261 - protein, 7 aa, Artificial Sequence
  synthetic peptide
  misc_feature (1)..(3): Xaa can be any naturally occurring amino acid
  PEPTIDE (7)..(7): C, S, or T
XXXCRLX

SEQ ID NO: 262 - protein, 5 aa, Artificial Sequence
  synthetic peptide
  misc_feature (1)..(1): Xaa can be any naturally occurring amino acid
XCRLC

SEQ ID NO: 263 - protein, 6 aa, Artificial Sequence
  synthetic peptide
  misc_feature (1)..(2): Xaa can be any naturally occurring amino acid
XXCRLC

SEQ ID NO: 264 - protein, 7 aa, Artificial Sequence
  synthetic peptide
  misc_feature (1)..(3): Xaa can be any naturally occurring amino acid
XXXCRLC

SEQ ID NO: 265 - protein, 5 aa, Artificial Sequence
  synthetic peptide
  misc_feature (1)..(1): Xaa can be any naturally occurring amino acid
XSRLC

SEQ ID NO: 266 - protein, 6 aa, Artificial Sequence
  synthetic peptide
  misc_feature (1)..(2): Xaa can be any naturally occurring amino acid
XXSRLC

SEQ ID NO: 267 - protein, 7 aa, Artificial Sequence
  synthetic peptide
  misc_feature (1)..(3): Xaa can be any naturally occurring amino acid
XXXSRLC

SEQ ID NO: 268 - protein, 5 aa, Artificial Sequence
  synthetic peptide
  misc_feature (1)..(1): Xaa can be any naturally occurring amino acid
XTRLC

SEQ ID NO: 269 - protein, 6 aa, Artificial Sequence
  synthetic peptide
  misc_feature (1)..(2): Xaa can be any naturally occurring amino acid
XXTRLC

SEQ ID NO: 270 - protein, 7 aa, Artificial Sequence
  synthetic peptide
  misc_feature (1)..(3): Xaa can be any naturally occurring amino acid
XXXTRLC

SEQ ID NO: 271 - protein, 5 aa, Artificial Sequence
  synthetic peptide
  misc_feature (1)..(1): Xaa can be any naturally occurring amino acid
XCRLC

SEQ ID NO: 272 - protein, 6 aa, Artificial Sequence
  synthetic peptide
  misc_feature (1)..(2): Xaa can be any naturally occurring amino acid
XXCRLC

SEQ ID NO: 273 - protein, 7 aa, Artificial Sequence
  synthetic peptide
  misc_feature (1)..(3): Xaa can be any naturally occurring amino acid
XXXCRLC

SEQ ID NO: 274 - protein, 5 aa, Artificial Sequence
  synthetic peptide
  misc_feature (1)..(1): Xaa can be any naturally occurring amino acid
XCRLS

SEQ ID NO: 275 - protein, 6 aa, Artificial Sequence
  synthetic peptide
  misc_feature (1)..(2): Xaa can be any naturally occurring amino acid
XXCRLS

SEQ ID NO: 276 - protein, 7 aa, Artificial Sequence
  synthetic peptide
  misc_feature (1)..(3): Xaa can be any naturally occurring amino acid
XXXCRLS

SEQ ID NO: 277 - protein, 5 aa, Artificial Sequence
  synthetic peptide
  misc_feature (1)..(1): Xaa can be any naturally occurring amino acid
XCRLT

SEQ ID NO: 278 - protein, 6 aa, Artificial Sequence
  synthetic peptide
  misc_feature (1)..(2): Xaa can be any naturally occurring amino acid
XXCRLT

SEQ ID NO: 279 - protein, 7 aa, Artificial Sequence
  synthetic peptide
  misc_feature (1)..(3): Xaa can be any naturally occurring amino acid
XXXCRLT

SEQ ID NO: 280 - protein, 5 aa, Artificial Sequence
  synthetic peptide
  misc_feature (1)..(1): Xaa can be any naturally occurring amino acid
  PEPTIDE (2)..(2): C, S, or T
XXHPC

SEQ ID NO: 281 - protein, 6 aa, Artificial Sequence
  synthetic peptide
  misc_feature (1)..(2): Xaa can be any naturally occurring amino acid
  PEPTIDE (3)..(3): C, S, or T
XXXHPC

SEQ ID NO: 282 - protein, 7 aa, Artificial Sequence
  synthetic peptide
  misc_feature (1)..(3): Xaa can be any naturally occurring amino acid
  PEPTIDE (4)..(4): C, S, or T
XXXXHPC

SEQ ID NO: 283 - protein, 5 aa, Artificial Sequence
  synthetic peptide
  misc_feature (1)..(1): Xaa can be any naturally occurring amino acid
  PEPTIDE (5)..(5): C, S, or T
XCHPX

SEQ ID NO: 284 - protein, 6 aa, Artificial Sequence
  synthetic peptide
  misc_feature (1)..(2): Xaa can be any naturally occurring amino acid
  PEPTIDE (6)..(6): C, S, or T
XXCHPX

SEQ ID NO: 285 - protein, 7 aa, Artificial Sequence
  synthetic peptide
  misc_feature (1)..(3): Xaa can be any naturally occurring amino acid
  PEPTIDE (7)..(7): C, S, or T
XXXCHPX

SEQ ID NO: 286 - protein, 5 aa, Artificial Sequence
  synthetic peptide
  misc_feature (1)..(1): Xaa can be any naturally occurring amino acid
XCHPC

SEQ ID NO: 287 - protein, 6 aa, Artificial Sequence
  synthetic peptide
  misc_feature (1)..(2): Xaa can be any naturally occurring amino acid
XXCHPC

SEQ ID NO: 288 - protein, 7 aa, Artificial Sequence
  synthetic peptide
  misc_feature (1)..(3): Xaa can be any naturally occurring amino acid
XXXCHPC

SEQ ID NO: 289 - protein, 5 aa, Artificial Sequence
  synthetic peptide
  misc_feature (1)..(1): Xaa can be any naturally occurring amino acid
XSHPC

SEQ ID NO: 290 - protein, 6 aa, Artificial Sequence
  synthetic peptide
  misc_feature (1)..(2): Xaa can be any naturally occurring amino acid
XXSHPC

SEQ ID NO: 291 - protein, 7 aa, Artificial Sequence
  synthetic peptide
  misc_feature (1)..(3): Xaa can be any naturally occurring amino acid
XXXSHPC

SEQ ID NO: 292 - protein, 5 aa, Artificial Sequence
  synthetic peptide
  misc_feature (1)..(1): Xaa can be any naturally occurring amino acid
XTHPC

SEQ ID NO: 293 - protein, 6 aa, Artificial Sequence
  synthetic peptide
  misc_feature (1)..(2): Xaa can be any naturally occurring amino acid
XXTHPC

SEQ ID NO: 294 - protein, 7 aa, Artificial Sequence
  synthetic peptide
  misc_feature (1)..(3): Xaa can be any naturally occurring amino acid
XXXTHPC

SEQ ID NO: 295 - protein, 5 aa, Artificial Sequence
  synthetic peptide
  misc_feature (1)..(1): Xaa can be any naturally occurring amino acid
XCHPC

SEQ ID NO: 296 - protein, 6 aa, Artificial Sequence
  synthetic peptide
  misc_feature (1)..(2): Xaa can be any naturally occurring amino acid
XXCHPC

SEQ ID NO: 297 - protein, 7 aa, Artificial Sequence
  synthetic peptide
  misc_feature (1)..(3): Xaa can be any naturally occurring amino acid
XXXCHPC

SEQ ID NO: 298 - protein, 5 aa, Artificial Sequence
  synthetic peptide
  misc_feature (1)..(1): Xaa can be any naturally occurring amino acid
XCHPS

SEQ ID NO: 299 - protein, 6 aa, Artificial Sequence
  synthetic peptide
  misc_feature (1)..(2): Xaa can be any naturally occurring amino acid
XXCHPS

SEQ ID NO: 300 - protein, 7 aa, Artificial Sequence
  synthetic peptide
  misc_feature (1)..(3): Xaa can be any naturally occurring amino acid
XXXCHPS

SEQ ID NO: 301 - protein, 5 aa, Artificial Sequence
  synthetic peptide
  misc_feature (1)..(1): Xaa can be any naturally occurring amino acid
XCHPT

SEQ ID NO: 302 - protein, 6 aa, Artificial Sequence
  synthetic peptide
  misc_feature (1)..(2): Xaa can be any naturally occurring amino acid
XXCHPT

SEQ ID NO: 303 - protein, 7 aa, Artificial Sequence
  synthetic peptide
  misc_feature (1)..(3): Xaa can be any naturally occurring amino acid
XXXCHPT

SEQ ID NO: 304 - protein, 21 aa, Artificial Sequence
  synthetic peptide
  misc_feature (1)..(1): Xaa can be any naturally occurring amino acid
XCPYCVQYIKANSKFIGITEL

SEQ ID NO: 305 - protein, 22 aa, Artificial Sequence
  synthetic peptide
  misc_feature (1)..(2): Xaa can be any naturally occurring amino acid
XXCPYCVQYIKANSKFIGITEL

SEQ ID NO: 306 - protein, 23 aa, Artificial Sequence
  synthetic peptide
  misc_feature (1)..(3): Xaa can be any naturally occurring amino acid
XXXCPYCVQYIKANSKFIGITEL

SEQ ID NO: 307 - protein, 4 aa, Artificial Sequence
  synthetic peptide
CPYC

SEQ ID NO: 308 - protein, 13 aa, Artificial Sequence
  synthetic peptide
QYIKANSKFIGIT

SEQ ID NO: 309 - protein, 21 aa, Artificial Sequence
  synthetic peptide
  misc_feature (1)..(1): Xaa can be any naturally occurring amino acid
XCHGCVQYIKANSKFIGITEL

SEQ ID NO: 310 - protein, 22 aa, Artificial Sequence
  synthetic peptide
  misc_feature (1)..(2): Xaa can be any naturally occurring amino acid
XXCHGCVQYIKANSKFIGITEL

SEQ ID NO: 311 - protein, 23 aa, Artificial Sequence
  synthetic peptide
  misc_feature (1)..(3): Xaa can be any naturally occurring amino acid
XXXCHGCVQYIKANSKFIGITEL

SEQ ID NO: 312 - protein, 4 aa, Artificial Sequence
  synthetic peptide
CHGC

SEQ ID NO: 313 - protein, 14 aa, Artificial Sequence
  synthetic peptide
  misc_feature (1)..(1): Xaa can be any naturally occurring amino acid
XCPYCGGFIGLMYY

SEQ ID NO: 314 - protein, 15 aa, Artificial Sequence
  synthetic peptide
  misc_feature (1)..(2): Xaa can be any naturally occurring amino acid
XXCPYCGGFIGLMYY

SEQ ID NO: 315 - protein, 16 aa, Artificial Sequence
  synthetic peptide
  misc_feature (1)..(3): Xaa can be any naturally occurring amino acid
XXXCPYCGGFIGLMYY

SEQ ID NO: 316 - protein, 7 aa, Artificial Sequence
  synthetic peptide
FIGLMYY

SEQ ID NO: 317 - protein, 20 aa, Artificial Sequence
  synthetic peptide
  misc_feature (1)..(1): Xaa can be any naturally occurring amino acid
XCPYCGWYRSPFSRVVHLYR

SEQ ID NO: 318 - protein, 21 aa, Artificial Sequence
  synthetic peptide
  misc_feature (1)..(2): Xaa can be any naturally occurring amino acid
XXCPYCGWYRSPFSRVVHLYR

SEQ ID NO: 319 - protein, 22 aa, Artificial Sequence
  synthetic peptide
  misc_feature (1)..(3): Xaa can be any naturally occurring amino acid
XXXCPYCGWYRSPFSRVVHLYR

SEQ ID NO: 320 - protein, 9 aa, Artificial Sequence
  synthetic peptide
YRSPFSRVV

SEQ ID NO: 321 - protein, 4 aa, Artificial Sequence
  synthetic peptide
HLYR

SEQ ID NO: 322 - protein, 17 aa, Artificial Sequence
  synthetic peptide
  misc_feature (1)..(1): Xaa can be any naturally occurring amino acid
XCPYCGWYRSPFSRVVK

SEQ ID NO: 323 - protein, 18 aa, Artificial Sequence
  synthetic peptide
  misc_feature (1)..(2): Xaa can be any naturally occurring amino acid
XXCPYCGWYRSPFSRVVK

SEQ ID NO: 324 - protein, 19 aa, Artificial Sequence
  synthetic peptide
  misc_feature (1)..(3): Xaa can be any naturally occurring amino acid
XXXCPYCGWYRSPFSRVVK

SEQ ID NO: 325 - protein, 18 aa, Artificial Sequence
  synthetic peptide
  misc_feature (1)..(1): Xaa can be any naturally occurring amino acid
XCPYCGWYRSPFSRVVKK

SEQ ID NO: 326 - protein, 19 aa, Artificial Sequence
  synthetic peptide
  misc_feature (1)..(2): Xaa can be any naturally occurring amino acid
XXCPYCGWYRSPFSRVVKK

SEQ ID NO: 327 - protein, 20 aa, Artificial Sequence
  synthetic peptide
  misc_feature (1)..(3): Xaa can be any naturally occurring amino acid
XXXCPYCGWYRSPFSRVVKK

SEQ ID NO: 328 - protein, 20 aa, Artificial Sequence
  synthetic peptide
  misc_feature (1)..(1): Xaa can be any naturally occurring amino acid
XCPYCVRYFLRVPSWKITLF

SEQ ID NO: 329 - protein, 21 aa, Artificial Sequence
  synthetic peptide
  misc_feature (1)..(2): Xaa can be any naturally occurring amino acid
XXCPYCVRYFLRVPSWKITLF

SEQ ID NO: 330 - protein, 22 aa, Artificial Sequence
  synthetic peptide
  misc_feature (1)..(3): Xaa can be any naturally occurring amino acid
XXXCPYCVRYFLRVPSWKITLF

SEQ ID NO: 331 - protein, 9 aa, Artificial Sequence
  synthetic peptide
FLRVPSWKI

SEQ ID NO: 332 - protein, 20 aa, Artificial Sequence
  synthetic peptide
  misc_feature (1)..(1): Xaa can be any naturally occurring amino acid
XCPYCSLQPLALEGSLQKRG

SEQ ID NO: 333 - protein, 21 aa, Artificial Sequence
  synthetic peptide
  misc_feature (1)..(2): Xaa can be any naturally occurring amino acid
XXCPYCSLQPLALEGSLQKRG

SEQ ID NO: 334 - protein, 22 aa, Artificial Sequence
  synthetic peptide
  misc_feature (1)..(3): Xaa can be any naturally occurring amino acid
XXXCPYCSLQPLALEGSLQKRG

SEQ ID NO: 335 - protein, 4 aa, Artificial Sequence
  synthetic peptide
SLQP

SEQ ID NO: 336 - protein, 9 aa, Artificial Sequence
  synthetic peptide
LALEGSLQK

SEQ ID NO: 337 - protein, 20 aa, Artificial Sequence
  synthetic peptide
WCPYCSLQPLALEGSLQKRG

SEQ ID NO: 338 - protein, 20 aa, Artificial Sequence
  synthetic peptide
PCPYCSLQPLALEGSLQKRG

SEQ ID NO: 339 - protein, 20 aa, Artificial Sequence
  synthetic peptide
CPYCVQYIKANSKFIGITEL

SEQ ID NO: 340 - protein, 21 aa, Artificial Sequence
  synthetic peptide
DCPYCVQYIKANSKFIGITEL

SEQ ID NO: 341 - protein, 21 aa, Artificial Sequence
  synthetic peptide
ECPYCVQYIKANSKFIGITEL

SEQ ID NO: 342 - protein, 21 aa, Artificial Sequence
  synthetic peptide
SCPYCVQYIKANSKFIGITEL

SEQ ID NO: 343 - protein, 21 aa, Artificial Sequence
  synthetic peptide
TCPYCVQYIKANSKFIGITEL

SEQ ID NO: 344 - protein, 21 aa, Artificial Sequence
  synthetic peptide
NCPYCVQYIKANSKFIGITEL

SEQ ID NO: 345 - protein, 21 aa, Artificial Sequence
  synthetic peptide
QCPYCVQYIKANSKFIGITEL

SEQ ID NO: 346 - protein, 21 aa, Artificial Sequence
  synthetic peptide
CCPYCVQYIKANSKFIGITEL

SEQ ID NO: 347 - protein, 21 aa, Artificial Sequence
  synthetic peptide
GCPYCVQYIKANSKFIGITEL

SEQ ID NO: 348 - protein, 21 aa, Artificial Sequence
  synthetic peptide
PCPYCVQYIKANSKFIGITEL

SEQ ID NO: 349 - protein, 21 aa, Artificial Sequence
  synthetic peptide
ACPYCVQYIKANSKFIGITEL

SEQ ID NO: 350 - protein, 21 aa, Artificial Sequence
  synthetic peptide
ICPYCVQYIKANSKFIGITEL

SEQ ID NO: 351 - protein, 21 aa, Artificial Sequence
  synthetic peptide
LCPYCVQYIKANSKFIGITEL

SEQ ID NO: 352 - protein, 21 aa, Artificial Sequence
  synthetic peptide
MCPYCVQYIKANSKFIGITEL

SEQ ID NO: 353 - protein, 21 aa, Artificial Sequence
  synthetic peptide
FCPYCVQYIKANSKFIGITEL

SEQ ID NO: 354 - protein, 21 aa, Artificial Sequence
  synthetic peptide
WCPYCVQYIKANSKFIGITEL

SEQ ID NO: 355 - protein, 21 aa, Artificial Sequence
  synthetic peptide
YCPYCVQYIKANSKFIGITEL

SEQ ID NO: 356 - protein, 21 aa, Artificial Sequence
  synthetic peptide
VCPYCVQYIKANSKFIGITEL

SEQ ID NO: 357 - protein, 20 aa, Artificial Sequence
  synthetic peptide
CHGCVQYIKANSKFIGITEL

SEQ ID NO: 358 - protein, 21 aa, Artificial Sequence
  synthetic peptide
DCHGCVQYIKANSKFIGITEL

SEQ ID NO: 359 - protein, 21 aa, Artificial Sequence
  synthetic peptide
ECHGCVQYIKANSKFIGITEL

SEQ ID NO: 360 - protein, 21 aa, Artificial Sequence
  synthetic peptide
SCHGCVQYIKANSKFIGITEL

SEQ ID NO: 361 - protein, 21 aa, Artificial Sequence
  synthetic peptide
TCHGCVQYIKANSKFIGITEL

SEQ ID NO: 362 - protein, 21 aa, Artificial Sequence
  synthetic peptide
NCHGCVQYIKANSKFIGITEL

SEQ ID NO: 363 - protein, 21 aa, Artificial Sequence
  synthetic peptide
QCHGCVQYIKANSKFIGITEL

SEQ ID NO: 364 - protein, 21 aa, Artificial Sequence
  synthetic peptide
CCHGCVQYIKANSKFIGITEL

SEQ ID NO: 365 - protein, 21 aa, Artificial Sequence
  synthetic peptide
GCHGCVQYIKANSKFIGITEL

SEQ ID NO: 366 - protein, 21 aa, Artificial Sequence
  synthetic peptide
PCHGCVQYIKANSKFIGITEL

SEQ ID NO: 367 - protein, 21 aa, Artificial Sequence
  synthetic peptide
ACHGCVQYIKANSKFIGITEL

SEQ ID NO: 368 - protein, 21 aa, Artificial Sequence
  synthetic peptide
ICHGCVQYIKANSKFIGITEL

SEQ ID NO: 369 - protein, 21 aa, Artificial Sequence
  synthetic peptide
LCHGCVQYIKANSKFIGITEL

SEQ ID NO: 370 - protein, 21 aa, Artificial Sequence
  synthetic peptide
MCHGCVQYIKANSKFIGITEL

SEQ ID NO: 371 - protein, 21 aa, Artificial Sequence
  synthetic peptide
FCHGCVQYIKANSKFIGITEL

SEQ ID NO: 372 - protein, 21 aa, Artificial Sequence
  synthetic peptide
WCHGCVQYIKANSKFIGITEL

SEQ ID NO: 373 - protein, 21 aa, Artificial Sequence
  synthetic peptide
YCHGCVQYIKANSKFIGITEL

SEQ ID NO: 374 - protein, 21 aa, Artificial Sequence
  synthetic peptide
VCHGCVQYIKANSKFIGITEL

SEQ ID NO: 375 - protein, 13 aa, Artificial Sequence
  synthetic peptide
CPYCGGFIGLMYY

SEQ ID NO: 376 - protein, 14 aa, Artificial Sequence
  synthetic peptide
DCPYCGGFIGLMYY

SEQ ID NO: 377 - protein, 14 aa, Artificial Sequence
  synthetic peptide
ECPYCGGFIGLMYY

SEQ ID NO: 378 - protein, 14 aa, Artificial Sequence
  synthetic peptide
SCPYCGGFIGLMYY

SEQ ID NO: 379 - protein, 14 aa, Artificial Sequence
  synthetic peptide
TCPYCGGFIGLMYY

SEQ ID NO: 380 - protein, 14 aa, Artificial Sequence
  synthetic peptide
NCPYCGGFIGLMYY

SEQ ID NO: 381 - protein, 14 aa, Artificial Sequence
  synthetic peptide
QCPYCGGFIGLMYY

SEQ ID NO: 382 - protein, 14 aa, Artificial Sequence
  synthetic peptide
CCPYCGGFIGLMYY

SEQ ID NO: 383 - protein, 14 aa, Artificial Sequence
  synthetic peptide
GCPYCGGFIGLMYY

SEQ ID NO: 384 - protein, 14 aa, Artificial Sequence
  synthetic peptide
PCPYCGGFIGLMYY

SEQ ID NO: 385 - protein, 14 aa, Artificial Sequence
  synthetic peptide
ACPYCGGFIGLMYY

SEQ ID NO: 386 - protein, 14 aa, Artificial Sequence
  synthetic peptide
ICPYCGGFIGLMYY

SEQ ID NO: 387 - protein, 14 aa, Artificial Sequence
  synthetic peptide
LCPYCGGFIGLMYY

SEQ ID NO: 388 - protein, 14 aa, Artificial Sequence
  synthetic peptide
MCPYCGGFIGLMYY

SEQ ID NO: 389 - protein, 14 aa, Artificial Sequence
  synthetic peptide
FCPYCGGFIGLMYY

SEQ ID NO: 390 - protein, 14 aa, Artificial Sequence
  synthetic peptide
WCPYCGGFIGLMYY

SEQ ID NO: 391 - protein, 14 aa, Artificial Sequence
  synthetic peptide
YCPYCGGFIGLMYY

SEQ ID NO: 392 - protein, 14 aa, Artificial Sequence
  synthetic peptide
VCPYCGGFIGLMYY

SEQ ID NO: 393 - protein, 19 aa, Artificial Sequence
  synthetic peptide
CPYCGWYRSPFSRVVHLYR

SEQ ID NO: 394 - protein, 20 aa, Artificial Sequence
  synthetic peptide
DCPYCGWYRSPFSRVVHLYR

SEQ ID NO: 395 - protein, 20 aa, Artificial Sequence
  synthetic peptide
ECPYCGWYRSPFSRVVHLYR

SEQ ID NO: 396 - protein, 20 aa, Artificial Sequence
  synthetic peptide
SCPYCGWYRSPFSRVVHLYR

SEQ ID NO: 397 - protein, 20 aa, Artificial Sequence
  synthetic peptide
TCPYCGWYRSPFSRVVHLYR

SEQ ID NO: 398 - protein, 20 aa, Artificial Sequence
  synthetic peptide
NCPYCGWYRSPFSRVVHLYR

SEQ ID NO: 399 - protein, 20 aa, Artificial Sequence
  synthetic peptide
QCPYCGWYRSPFSRVVHLYR

SEQ ID NO: 400 - protein, 20 aa, Artificial Sequence
  synthetic peptide
CCPYCGWYRSPFSRVVHLYR

SEQ ID NO: 401 - protein, 20 aa, Artificial Sequence
  synthetic peptide
GCPYCGWYRSPFSRVVHLYR

SEQ ID NO: 402 - protein, 20 aa, Artificial Sequence
  synthetic peptide
PCPYCGWYRSPFSRVVHLYR

SEQ ID NO: 403 - protein, 20 aa, Artificial Sequence
  synthetic peptide
ACPYCGWYRSPFSRVVHLYR

SEQ ID NO: 404 - protein, 20 aa, Artificial Sequence
  synthetic peptide
ICPYCGWYRSPFSRVVHLYR

SEQ ID NO: 405 - protein, 20 aa, Artificial Sequence
  synthetic peptide
LCPYCGWYRSPFSRVVHLYR

SEQ ID NO: 406 - protein, 20 aa, Artificial Sequence
  synthetic peptide
MCPYCGWYRSPFSRVVHLYR

SEQ ID NO: 407 - protein, 20 aa, Artificial Sequence
  synthetic peptide
FCPYCGWYRSPFSRVVHLYR

SEQ ID NO: 408 - protein, 20 aa, Artificial Sequence
  synthetic peptide
WCPYCGWYRSPFSRVVHLYR

SEQ ID NO: 409 - protein, 20 aa, Artificial Sequence
  synthetic peptide
YCPYCGWYRSPFSRVVHLYR

SEQ ID NO: 410 - protein, 20 aa, Artificial Sequence
  synthetic peptide
VCPYCGWYRSPFSRVVHLYR

SEQ ID NO: 411 - protein, 18 aa, Artificial Sequence
  synthetic peptide
CGCGWYRSPFSRVVHLYR

SEQ ID NO: 412 - protein, 19 aa, Artificial Sequence
  synthetic peptide
DCGCGWYRSPFSRVVHLYR

SEQ ID NO: 413 - protein, 19 aa, Artificial Sequence
  synthetic peptide
ECGCGWYRSPFSRVVHLYR

SEQ ID NO: 414 - protein, 19 aa, Artificial Sequence
  synthetic peptide
SCGCGWYRSPFSRVVHLYR

SEQ ID NO: 415 - protein, 19 aa, Artificial Sequence
  synthetic peptide
TCGCGWYRSPFSRVVHLYR

SEQ ID NO: 416 - protein, 19 aa, Artificial Sequence
  synthetic peptide
NCGCGWYRSPFSRVVHLYR

SEQ ID NO: 417 - protein, 19 aa, Artificial Sequence
  synthetic peptide
QCGCGWYRSPFSRVVHLYR

SEQ ID NO: 418 - protein, 19 aa, Artificial Sequence
  synthetic peptide
CCGCGWYRSPFSRVVHLYR

SEQ ID NO: 419 - protein, 19 aa, Artificial Sequence
  synthetic peptide
GCGCGWYRSPFSRVVHLYR

SEQ ID NO: 420 - protein, 19 aa, Artificial Sequence
  synthetic peptide
PCGCGWYRSPFSRVVHLYR

SEQ ID NO: 421 - protein, 19 aa, Artificial Sequence
  synthetic peptide
ACGCGWYRSPFSRVVHLYR

SEQ ID NO: 422 - protein, 19 aa, Artificial Sequence
  synthetic peptide
ICGCGWYRSPFSRVVHLYR

SEQ ID NO: 423 - protein, 19 aa, Artificial Sequence
  synthetic peptide
LCGCGWYRSPFSRVVHLYR

SEQ ID NO: 424 - protein, 19 aa, Artificial Sequence
  synthetic peptide
MCGCGWYRSPFSRVVHLYR

SEQ ID NO: 425 - protein, 19 aa, Artificial Sequence
  synthetic peptide
FCGCGWYRSPFSRVVHLYR

SEQ ID NO: 426 - protein, 19 aa, Artificial Sequence
  synthetic peptide
WCGCGWYRSPFSRVVHLYR

SEQ ID NO: 427 - protein, 19 aa, Artificial Sequence
  synthetic peptide
YCGCGWYRSPFSRVVHLYR

SEQ ID NO: 428 - protein, 19 aa, Artificial Sequence
  synthetic peptide
VCGCGWYRSPFSRVVHLYR

SEQ ID NO: 429 - protein, 20 aa, Artificial Sequence
  synthetic peptide
WCPYCSLQPLALEGSLQKRG

SEQ ID NO: 430 - protein, 20 aa, Artificial Sequence
  synthetic peptide
PCPYCSLQPLALEGSLQKRG

SEQ ID NO: 431 - protein, 20 aa, Artificial Sequence
  synthetic peptide
YCPYCSLQPLALEGSLQKRG

SEQ ID NO: 432 - protein, 20 aa, Artificial Sequence
  synthetic peptide
FCPYCSLQPLALEGSLQKRG

SEQ ID NO: 433 - protein, 20 aa, Artificial Sequence
  synthetic peptide
VCPYCSLQPLALEGSLQKRG

SEQ ID NO: 434 - protein, 20 aa, Artificial Sequence
  synthetic peptide
ICPYCSLQPLALEGSLQKRG

SEQ ID NO: 435 - protein, 20 aa, Artificial Sequence
  synthetic peptide
MCPYCSLQPLALEGSLQKRG

SEQ ID NO: 436 - protein, 20 aa, Artificial Sequence
  synthetic peptide
CCPYCSLQPLALEGSLQKRG

SEQ ID NO: 437 - protein, 20 aa, Artificial Sequence
  synthetic peptide
LCPYCSLQPLALEGSLQKRG

SEQ ID NO: 438 - protein, 20 aa, Artificial Sequence
  synthetic peptide
GCPYCSLQPLALEGSLQKRG

SEQ ID NO: 439 - protein, 20 aa, Artificial Sequence
  synthetic peptide
ACPYCSLQPLALEGSLQKRG

SEQ ID NO: 440 - protein, 20 aa, Artificial Sequence
  synthetic peptide
QCPYCSLQPLALEGSLQKRG

SEQ ID NO: 441 - protein, 20 aa, Artificial Sequence
  synthetic peptide
TCPYCSLQPLALEGSLQKRG

SEQ ID NO: 442 - protein, 20 aa, Artificial Sequence
  synthetic peptide
NCPYCSLQPLALEGSLQKRG

SEQ ID NO: 443 - protein, 20 aa, Artificial Sequence
  synthetic peptide
SCPYCSLQPLALEGSLQKRG

SEQ ID NO: 444 - protein, 20 aa, Artificial Sequence
  synthetic peptide
ECPYCSLQPLALEGSLQKRG

SEQ ID NO: 445 - protein, 20 aa, Artificial Sequence
  synthetic peptide
DCPYCSLQPLALEGSLQKRG

SEQ ID NO: 446 - protein, 20 aa, Artificial Sequence
  synthetic peptide
HCPYCSLQPLALEGSLQKRG

SEQ ID NO: 447 - protein, 5 aa, Artificial Sequence
  synthetic peptide
HCPYC

SEQ ID NO: 448 - protein, 21 aa, Artificial Sequence
  synthetic peptide
  PEPTIDE (1)..(1): any amino acid or non-natural amino acid excluding R, K, H, D, E, and A
XCPYCVRYFLRVPSWKITLFK

SEQ ID NO: 449 - protein, 22 aa, Artificial Sequence
  synthetic peptide
  PEPTIDE (1)..(1): any amino acid or non-natural amino acid excluding R, K, H, D, E, and A
  misc_feature (2)..(2): Xaa can be any naturally occurring amino acid
XXCPYCVRYFLRVPSWKITLFK

SEQ ID NO: 450 - protein, 23 aa, Artificial Sequence
  synthetic peptide
  PEPTIDE (1)..(1): any amino acid or non-natural amino acid excluding R, K, H, D, E, and A
  misc_feature (2)..(3): Xaa can be any naturally occurring amino acid
XXXCPYCVRYFLRVPSWKITLFK

(19) United States
(12) Patent Application Publication
(10) Pub. No.: US 2024/0189404 A1
(43) Pub. Date: Jun. 13, 2024

(54) IMMUNOGENIC PEPTIDES WITH EXTENDED OXIDOREDUCTASE MOTIFS

(71) Applicant: IMCYSE SA, Angleur (Liége) (BE)

(72) Inventor: Milos ERAK, Bruxelles (BE)

(21) Appl. No.: 17/923,117

(22) PCT Filed: May 6, 2021
(86) PCT No.: PCT/EP2021/062002

§ 371 Date: Nov. 3, 2022

(30) Foreign Application Priority Data
May 6, 2020 (EP) 20173252.6

(51) Int. Cl. A61K 39/00; A61K 35/17; C07K 14/74; C12N 5/0783; C12N 9/02

(52) U.S. Cl. CPC A61K 39/001111; A61K 35/17; A61K 39/4611; C07K 14/70539; C12N 5/0638; C12N 9/0004; C07K 2319/40

(57) ABSTRACT

The invention relates to immunogenic peptides comprising MHC class II T-cell epitopes and oxidoreductase motifs with increased activity, and their use in regulating the immune response in subjects.

### CROSS-REFERENCE TO RELATED APPLICATIONS

This application is the U.S. national phase of International Application No. PCT/EP2021/062002 filed May 6, 2021 which designated the U.S. and claims priority to EP Patent Application No. 20173252.6 filed May 6, 2020, the entire contents of each of which are hereby incorporated by reference.

### BACKGROUND OF THE INVENTION

Several strategies have been described to prevent the generation of an unwanted immune response against an antigen. WO2008/017517 describes a new strategy using peptides comprising an MHC class II antigen of a given antigenic protein and an oxidoreductase motif. These peptides convert CD4+ T cells into a cell type with cytolytic properties called cytolytic CD4+ T cells. These cells are capable to kill via triggering apoptosis those antigen presenting cells (APC), which present the antigen from which the peptide is derived. WO2008/017517 demonstrates this concept for allergies and auto-immune diseases such as type I diabetes. Herein insulin can act as an auto-antigen.

WO2009101207 and Carlier et al. (2012) Plos one 7, 10 e45366 further describe the antigen specific cytolytic CD4+ cells in more detail.

WO2016059236 discloses further modified peptides comprising an MHC class II epitope wherein an additional Histidine is present in the proximity of the oxidoreductase motif. WO2018162498 discloses a peptide comprising a HCPYC oxidoreductase motif and an insulin MHC class II T cell epitope for the treatment of diabetes.

Both strategies are building upon the use of 4 amino acid oxidoreductase motifs of the [CST]XXC (SEQ ID NO: 1) or CXX[CST] (SEQ ID NO: 2) type, wherein C represents a cysteine residue, [CST] represents any one of a cysteine, serine or threonine residue and X represents any amino acid residue. In order to improve the efficacy of a treatment using such immunogenic peptides, the search for more active peptides and/or more potent oxidoreductase motifs continues.

### SUMMARY OF THE INVENTION

The present invention provides novel immunogenic peptides comprising an MHC class II T-cell epitope of an antigen and an oxidoreductase amino acid motif, where any amino acid which is not a basic amino acid such as R, K and H, and which is not A, D and E can be present immediately N- or C-terminal to said motif or indirectly adjacent to the N- or C-terminus of said motif by the occurrence of one or more additional amino acids.

The present invention relates to the following aspects:

Aspect 1: An immunogenic peptide, said immunogenic peptide comprising:
a) an oxidoreductase amino acid motif,
b) a T-cell epitope of an antigenic protein, and
c) a linker between a) and b) of between 0 and 7 amino acids
wherein: said oxidoreductase motif has the following sequence:

(SEQ ID NOs: 96-109)
Z(B)ₙ[CST]XₘC-
or
(SEQ ID NOs; 110-123)
Z(B)ₙCXₘ[CST]-;

wherein Z is any amino acid or non-natural amino acid, preferably excluding basic amino acids such as: R (Arginine), K (Lysine) and H (Histidine), and preferably excluding amino acids D (Aspartate), E (Glutamate), and/or A (Alanine);
wherein (B) is any amino acid;
wherein n is an integer of 0 to 2;
wherein X is any amino acid;
wherein m is an integer of 0 to 4, preferably wherein m is 1, 2, or 3, more preferably wherein m is 2;
wherein the hyphen (-) in said oxidoreductase motif indicates the point of attachment of the oxidoreductase motif to the N-terminal end of the linker (c) or the epitope (b), or to the C-terminal end of the linker (c) or the epitope (b).

In one embodiment, Z is not W.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Aspect 2: The immunogenic peptide according to aspect 1,
wherein in said oxidoreductase motif:
Z is any amino acid or non-natural amino acid excluding basic amino acids such as R (Arginine), K (Lysine) and H (Histidine), and excluding amino acids A (Alanine), D (Aspartate), and/or E (Glutamate); and wherein said T cell epitope is an MHC class II epitope.

In one embodiment, immunogenic peptides that comprise an epitope that is both an NKT and MHC class II T-cell epitope are excluded from the invention.

In preferred embodiments, Z is selected from the group consisting of: W, G, S, T, C, V, L, I, M, P, F, Y, N, and Q, most preferably Z is P, W or G.

In one embodiment, Z is not W.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Aspect 3: The immunogenic peptide according to aspect 1 or 2, wherein Z is selected from the group comprising amino acids: G (Glycine), I (Isoleucine), L (Leucine), P (Proline), and V (Valine).

Aspect 4: The immunogenic peptide according to aspect 1 or 2, wherein Z is selected from the group comprising amino acids: W (Tryptophan), F (Phenylalanine) and Y (Tyrosine).

Aspect 5: The immunogenic peptide according to aspect 1 or 2, wherein Z is selected from the group comprising amino acids: S (Serine) and T (Threonine).

Aspect 6: The immunogenic peptide according to aspect 1 or 2, wherein Z is M (Methionine).

Aspect 7: The immunogenic peptide according to aspect 1 or 2, wherein Z is selected from the group comprising amino acids: N (Asparagine) and Q (Glutamine).

Aspect 8: The immunogenic peptide according to any one of aspects 1 to 7, wherein X is any amino acid except for C (Cysteine), S (Serine), or T (Threonine).

Aspect 9: The immunogenic peptide according to any one of aspects 1 to 8, wherein one or more X is a basic amino acid, preferably wherein said one X is R, such as in motifs according to aspect 1 or 2, of any one of the following formulas: Z(B)ₙCRC (SEQ ID NOs: 3-5), Z(B)ₙCRXC (SEQ ID NOs: 6-8) or Z(B)ₙCRXXC (SEQ ID NOs: 9-11).

Aspect 10: The immunogenic peptide according to aspect 1, wherein said T cell epitope of an antigenic protein is an NKT cell epitope.

Aspect 11: The immunogenic peptide according to aspect 10, wherein said epitope has a length of between 7 and 30 amino acids, preferably between 7 and 25 amino acids, more preferably between 7 and 20 amino acids.

Aspect 12: The immunogenic peptide according to aspects 10 or 11, having a length of between 10 and 50 amino acids, preferably between 10 and 40 amino acids, more preferably between 10 and 30 amino acids, such as having a length of between 11 and 50 amino acids, preferably between 11 and 40 amino acids, more preferably between 11 and 30 amino acids.

Aspect 13: The immunogenic peptide according to any one of aspects 2 to 9, wherein said epitope has a length of between 9 and 30 amino acids, preferably between 9 and 25 amino acids, more preferably between 9 and 20 amino acids.

Aspect 14: The immunogenic peptide according to any one of aspects 2 to 9, or 13, having a length of between 12 and 50 amino acids, preferably between 12 and 40 amino acids, more preferably between 12 and 30 amino acids.

Aspect 15: The immunogenic peptide according to any one of aspects 1 to 14, wherein said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen.

Aspect 16: The immunogenic peptide according to any one of aspects 1 to 15, wherein the linker is of between 0 and 4 amino acids.

Aspect 17: The immunogenic peptide according to any one of aspects 1 to 16, wherein said oxidoreductase motif does not naturally occur within a region of 11 amino acids N-terminally or C-terminally of the T-cell epitope in said antigenic protein.

Aspect 18: The immunogenic peptide according to any one of aspects 1 to 17, wherein the T-cell epitope does not naturally comprise said oxidoreductase motif.

Aspect 19: The immunogenic peptide according to any one of aspects 2 to 9, or 13 to 18, wherein at least one X in the motif is P or Y, or wherein said oxidoreductase motif is selected from the group comprising: Z(B)ₙCPYC (SEQ ID NOs: 12 to 14); Z(B)ₙCGHC (SEQ ID NOs: 15 to 17); Z(B)ₙCHGC (SEQ ID NOs: 18 to 20); Z(B)ₙCRLC (SEQ ID NOs: 21 to 23); Z(B)ₙCGFC (SEQ ID NOs: 24 to 26); Z(B)ₙCHPC (SEQ ID NOs: 27 to 29); Z(B)ₙCGPC (SEQ ID NOs: 30 to 32); Z(B)ₙCC (SEQ ID NOs: 33 to 35); Z(B)ₙCRC (SEQ ID NOs: 36 to 38); Z(B)ₙCKC (SEQ ID NOS: 39 to 41); Z(B)ₙCRPYC (SEQ ID NOs: 42 to 44); Z(B)ₙCKPYC (SEQ ID NOs: 45 to 47); Z(B)ₙCRGHC (SEQ ID NOs: 48 to 50); Z(B)ₙCKGHC (SEQ ID NOs: 51 to 53); Z(B)ₙCRHGC (SEQ ID NOs: 54 to 56); Z(B)ₙCKHGC (SEQ ID NOs: 57 to 59); Z(B)ₙCRRLC (SEQ ID NOs: 60 to 62); Z(B)ₙCKRLC (SEQ ID NOs: 63 to 65); Z(B)ₙCRGFC (SEQ ID NOs: 66 to 68); Z(B)ₙCKGFC (SEQ ID NOs: 69 to 71); Z(B)ₙCRHPC (SEQ ID NOs: 72 to 74); Z(B)ₙCKHPC (SEQ ID NOs: 75 to 77); Z(B)ₙCRGPC (SEQ ID NOs: 78 to 80); and Z(B)ₙCKGPC (SEQ ID NOs: 81 to 83).

Aspect 20: In a preferred embodiment any one of aspects 2 to 9, or 13 to 19, the immunogenic peptide comprises any one of the following sequences:

(SEQ ID NOs: 84 to 86)
Z(B)ₙ-CPYC-GW-YRSPFSRVV-HLYR,
(SEQ ID NOs: 87 to 89)
Z(B)ₙ-CPYC-GW-YRSPFSRVV-K,
and
(SEQ ID NOs: 90 to 92)
Z(B)ₙ-CPYC-VRY-FLRVPSWKI-TLF,
(SEQ ID NOs: 93 to 95)
Z(B)ₙ-CPYC-SLQP-LALEGSLQK-RG,

more preferably wherein in any one of said sequences Z(B)ₙ is selected from: W, P, G, KW, KP, KG, RW, RP, RG, HW, HP, HG, PH, WH, GH, PK, WK, GK, PR, WR, and GR.

Aspect 21: A nucleic acid encoding the immunogenic peptide according to any one of aspects 1 to 20, preferably selected from isolated deoxyribonucleic acid (DNA), plasmid DNA (pDNA), coding DNA (cDNA), ribonucleic acid (RNA), messenger RNA (mRNA) or modified versions thereof. In some embodiments, said nucleic acid can be part of an expression cassette, optionally incorporated in a (viral) vector or plasmid that can be used for gene-therapy or can be present in the form of encapsulated or naked DNA or RNA to be administered according to techniques known in the pharmaceutical and gene therapeutic field.

Aspect 22: The immunogenic peptide according to any one of aspects 1 to 20, or the nucleic acid according to aspect 21, for use in medicine, more particularly for use in treating and/or prevention of an autoimmune disease, an infection with an intracellular pathogen, a tumor, an allograft rejection, or an immune response to a soluble allofactor, to an allergen exposure or to a viral vector used for gene therapy or gene vaccination.

More particularly, the invention provides for the immunogenic peptide according to any one of aspects 2 to 20, or a polynucleotide encoding such an immunogenic peptide, for use in treating and/or preventing type 1 diabetes, wherein said MHC class II T-cell epitope is an epitope of (pro)insulin, glutamic acid decarboxylase 65 (GAD65), insulinoma antigen-2 (IA-2), heat shock protein (HSP), islet-specific glucose-6-phosphatase catalytic subunit related protein (IGRP), imogen-38 zinc transporter-8 (ZnT8), pancreatic duodenal homeobox factor 1 (PDX1), chromogranin A (CHGA), and islet amyloid polypeptide (IAPP). In a preferred embodiment, said epitope has a minimal length of 9 amino acids, preferably between 9 and 30, such as between 9 and 25 or between 9 and 20 amino acids.

More particularly, said immunogenic peptide comprises the following sequence: Z(B)ₙ-CPYC-SLQP-LALEGSLOK-RG, wherein Z(B)ₙ is selected from: W, P, G, KW, KP, KG, RW, RP, RG, HW, HP, HG, PH, WH, GH, PK, WK, GK, PR, WR, and GR.

More particularly, the invention provides for the immunogenic peptide according to any one of claims 2 to 20, or a polynucleotide encoding such an immunogenic peptide, for use in treating or preventing demyelinating disorders caused or aggravated by Myelin Oligodendrocyte Glycoprotein (MOG) auto-antigens and/or anti-MOG antibodies, more preferably selected from the group consisting of: Multiple Sclerosis (MS), Neuromyelitis Optica (NMO), Optic Neuritis, Acute Disseminated Encephalomyelitis, Transverse Myelitis, Adrenoleukodystrophy, Vanishing White Matter Disease, and Rubella induced mental retardation, wherein said MHC class II T-cell epitope is an epitope of the Myelin Oligodendrocyte Glycoprotein (MOG) auto-antigen. In a preferred embodiment, said epitope has a minimal length of 9 amino acids, preferably between 9 and 30, such as between 9 and 25 or between 9 and 20 amino acids.

More particularly, said immunogenic peptide comprises the sequence: Z(B)ₙ-CPYC-VRY-FLRVPSWKI-TLF (SEQ ID NOs: 90 to 92), Z(B)ₙ-CPYC-VRY-FLRVPSWKI-TLFK (SEQ ID NOS: 448 to 450), or Z(B)ₙ-CPYC-VRY-FLRVPSWKI-TLFKK (SEQ ID NOs: 124 to 126), wherein Z(B)ₙ is selected from: W, P, G, KW, KP, KG, RW, RP, RG, HW, HP, HG, PH, WH, GH, PK, WK, GK, PR, WR, and GR.

Aspect 23: A method for preparing an immunogenic peptide according to any one of aspects 1 to 22, comprising the steps of:
(a) providing a peptide sequence consisting of a T-cell epitope of said antigenic protein, and
(b) linking to said peptide sequence the oxidoreductase motif, such that said motif and said epitope are either adjacent to each other or separated by a linker of at most 7 amino acids.

Aspect 24: A method for obtaining a population of antigen-specific cytolytic CD4+ T cells, against APC presenting said antigen, the method comprising the steps of:
providing peripheral blood cells;
contacting said cells with an immunogenic peptide according to any one of aspects 1 to 20, or with the nucleic acid according to aspect 21, said peptide more particularly comprising:
a) an oxidoreductase amino acid motif,
b) a T-cell epitope of an antigenic protein, and
c) a linker between a) and b) of between 0 and 7 amino acids wherein: said oxidoreductase motif has the following sequence:
Z(B)ₙ[CST]XₘC- (SEQ ID NOs: 96-109) or Z(B)ₙCXₘ[CST]- (SEQ ID NOs: 110-123);
wherein Z is any amino acid or non-natural amino acid, preferably excluding basic amino acids such as; R (Arginine), K (Lysine) and H (Histidine), and preferably excluding amino acids D (Aspartate), E (Glutamate), and/or A (Alanine);
wherein (B) is any amino acid;
wherein n is an integer of 0 to 2;
wherein X is any amino acid;
wherein m is an integer of 0 to 4, preferably wherein m is 1, 2, or 3, more preferably wherein m is 2;
wherein the hyphen (-) in said oxidoreductase motif indicates the point of attachment of the oxidoreductase motif to the N-terminal end of the linker (c) or the epitope (b), or to the C-terminal end of the linker (c) or the epitope (b); and
expanding said cells in the presence of IL-2.

Aspect 25: The method according to aspect 24, wherein in said oxidoreductase motif, Z is any amino acid or non-natural amino acid excluding basic amino acids such as R (Arginine), K (Lysine) and H (Histidine), and excluding amino acids A (Alanine), D (Aspartate), and/or E (Glutamate); and wherein said T cell epitope is an MHC class II epitope.

In preferred embodiments, Z is selected from the group consisting of: W, G, S, T, C, V, L, I, M, P, F, Y, N, and Q, preferably wherein Z is W, G and P.

In one embodiment, immunogenic peptides that comprise an epitope that is both an NKT and MHC class II T-cell epitope are excluded from the invention.

In one embodiment, Z is not W.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Aspect 26: A method for obtaining a population of antigen-specific NKT cells, the method comprising the steps of:
providing peripheral blood cells;
contacting said cells with an immunogenic peptide according to any one of aspects 1, 3 to 11, 13 to 20, or the nucleic acid encoding said peptide, more particularly said peptide comprising:
a) an oxidoreductase amino acid motif,
b) an NKT-cell epitope of an antigenic protein, and
c) a linker between a) and b) of between 0 and 7 amino acids
   wherein: said oxidoreductase motif has the following sequence:

(SEQ ID NOs: 96-109)
Z(B)ₙ[CST]XₘC-
or
(SEQ ID NOs: 110-123)
Z(B)ₙCXₘ[CST]-;

wherein Z is any amino acid or non-natural amino acid preferably excluding basic amino acids such as; R (Arginine), K (Lysine) and H (Histidine), and preferably excluding amino acids D (Aspartate), E (Glutamate), and/or A (Alanine) and corresponds to the N- or C-terminal end of the immunogenic peptide;
wherein (B) is any amino acid wherein n is an integer of 0 to 2;
wherein m is an integer of 1 to 4, preferably wherein m is 1, 2, or 3, more preferably wherein m is 2;
wherein X is any amino acid;
wherein the hyphen (-) in said oxidoreductase motif indicates the point of attachment of the oxidoreductase motif to the N-terminal end of the linker (c) or the epitope (b), or to the C-terminal end of the linker (c) or the epitope (b)
and
expanding said cells in the presence of IL-2.

In preferred embodiments, Z is selected from the group consisting of: G, S, T, C, V, L, I, M, P, F, Y, N, and Q, more preferably wherein Z is P, W, or G.

In one embodiment, Z is not W.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Aspect 27: A method for obtaining a population of antigen-specific cytolytic CD4+ T cells, against APC presenting said antigen, the method comprising the steps of:
providing an immunogenic peptide according to any one of aspects 1 to 9 or 13 to 20 or a polynucleotide encoding said peptide, more particularly comprising:
a) an oxidoreductase amino acid motif,
b) an MHC class II T-cell epitope of an antigenic protein, and
c) a linker between a) and b) of between 0 and 7 amino acids
   wherein: said oxidoreductase motif has the following sequence:

(SEQ ID NOs: 96-109)
Z(B)ₙ[CST]XₘC-
or
(SEQ ID NOs: 110-123)
Z(B)ₙCXₘ[CST]-;

wherein Z is any amino acid or non-natural amino acid, preferably excluding basic amino acids such as; R (Arginine), K (Lysine) and H (Histidine), and preferably excluding amino acids D (Aspartate), E (Glutamate), and/or A (Alanine);
wherein (B) is any amino acid;
wherein n is an integer of 0 to 2;
wherein X is any amino acid;
wherein m is an integer of 0 to 4, preferably wherein m is 1, 2, or 3, more preferably wherein m is 2;
wherein the hyphen (-) in said oxidoreductase motif indicates the point of attachment of the oxidoreductase motif to the N-terminal end of the linker (c) or the epitope (b), or to the C-terminal end of the linker (c) or the epitope (b);
administering said peptide to a subject; and
obtaining said population of antigen-specific cytolytic CD4+ T cells from said subject.

In preferred embodiments, Z is selected from the group consisting of: W, G, S, T, C, V, L, I, M, P, F, Y, N, and Q, more preferably wherein Z is G, W, or P.

In one embodiment, immunogenic peptides that comprise an epitope that is both an NKT and MHC class II T-cell epitope are excluded from the invention.

In one embodiment, Z is not W.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Aspect 28: A method for obtaining a population of antigen-specific NKT cells, the method comprising the steps of:
providing an immunogenic peptide according to any one of aspects 1, 3 to 11, 13 to 20, or the nucleic acid encoding said peptide, more particularly comprising:
a) an oxidoreductase motif,
b) an NKT-cell epitope of an antigenic protein, and
c) a linker between a) and b) of between 0 and 7 amino acids
   wherein Z is any amino acid or non-natural amino acid, excluding basic amino acids such as; R (Arginine), K (Lysine) and H (Histidine), and excluding amino acids D (Aspartate), E (Glutamate), and/or A (Alanine);
   wherein (B) is any amino acid;
   wherein n is an integer of 0 to 2;
   wherein X is any amino acid;
   wherein m is an integer of 0 to 4, preferably wherein m is 1, 2, or 3, more preferably wherein m is 2;
   wherein the hyphen (-) in said oxidoreductase motif indicates the point of attachment of the oxidoreductase motif to the N-terminal end of the linker (c) or the epitope (b), or to the C-terminal end of the linker (c) or the epitope (b);
   wherein X is any amino acid;
wherein (B) is any amino acid;
administering said peptide to a subject; and
obtaining said population of antigen-specific NKT cells from said subject.

In preferred embodiments, Z is selected from the group consisting of: G, S, T, C, V, L, I, M, P, F, Y, D, E, N, and Q, more preferably wherein Z is G, W, or P.

In one embodiment, Z is not W.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Aspect 29: The population of antigen-specific cytolytic CD4+ T cells or NKT cells obtainable by the method of aspects 24 to 29 for use in the treatment and/or prevention of an autoimmune disease, an infection with an intracellular pathogen, a tumor, an allograft rejection, or an immune response to a soluble allofactors, to an allergen exposure or to a viral vector used for gene therapy or gene vaccination.

Aspect 30: A method of treating and/or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of administering the immunogenic peptide according to any one of aspects 1 to 20, the nucleic acid according to aspect 21 or the cell population according to aspect 29 to said individual.

Aspect 31: A method of treating or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of:
providing peripheral blood cells of said individual,
contacting said cells with an antigenic peptide according to any of aspects 1 to 20, or the nucleic acid according to aspect 21,
expanding said cells, and
administering said expanded cells to said individual.

Aspect 32: A method of treating type 1 diabetes in a (human) subject, comprising administering a therapeutically effective amount of an immunogenic peptide according to any one of aspects 1 to 20 or a polynucleotide encoding such an immunogenic peptide, wherein said T-cell epitope is an MHC class II epitope of (pro)insulin, glutamic acid decarboxylase 65 (GAD65), insulinoma antigen-2 (IA-2), heat shock protein (HSP), islet-specific glucose-6-phosphatase catalytic subunit related protein (IGRP), imogen-38 zinc transporter-8 (ZnT8), pancreatic duodenal homeobox factor 1 (PDX1), chromogranin A (CHGA), and islet amyloid polypeptide (IAPP). In a preferred embodiment, said epitope has a minimal length of 9 amino acids, preferably between 9 and 30, such as between 9 and 25 or between 9 and 20 amino acids.

More specifically, said immunogenic peptide comprises the following sequence: Z(B)ₙ-CPYC-SLQP-LALEGSLOK-RG (SEQ ID NOs: 93-95), wherein Z(B)ₙ is selected from: W, P, G, KW, KP, KG, RW, RP, RG, HW, HP, HG, PH, WH, GH, PK, WK, GK, PR, WR, and GR.

Aspect 33: A method of treating or preventing demyelinating disorders caused or aggravated by Myelin Oligodendrocyte Glycoprotein (MOG) auto-antigens and/or anti-MOG antibodies in a (human) subject, comprising administering a therapeutically effective amount of an immunogenic peptide according to any one of aspects 1 to 20 or a polynucleotide encoding such an immunogenic peptide, wherein said T-cell epitope is an MHC class II epitope of Myelin Oligodendrocyte Glycoprotein (MOG). In a preferred embodiment, said epitope has a minimal length of 9 amino acids, preferably between 9 and 30, such as between 9 and 25 or between 9 and 20 amino acids.

More preferably selected from the group consisting of: Multiple Sclerosis (MS), Neuromyelitis Optica (NMO), Optic Neuritis, Acute Disseminated Encephalomyelitis, Transverse Myelitis, Adrenoleukodystrophy, Vanishing White Matter Disease, and Rubella induced mental retardation More preferably, said immunogenic peptide comprises the sequence: Z(B)ₙ-CPYC-VRY-FLRVPSWKI-TLF (SEQ ID NOs: 90-92), Z(B)ₙ-CPYC-VRY-FLRVPSWKI-TLFK (SEQ ID NOS: 448 to 450), or Z(B)ₙ-CPYC-VRY-FLRVPSWKI-TLFKK (SEQ ID NOs: 124-126), wherein Z(B)ₙ is selected from: W, P, G, KW, KP, KG, RW, RP, RG, HW, HP, HG, PH, WH, GH, PK, WK, GK, PR, WR, and GR.

In one embodiment of any one of the above aspects, immunogenic peptides that comprise an epitope that is both an NKT and MHC class II T-cell epitope are excluded from the invention.

In a preferred embodiment of any one of said aspects, the linker comprises at least 1 amino acid, at least 2 amino acids, at least 3 amino acids, or at least 4 amino acids. Preferably, said linker comprises between 1 and 7 amino acids, such as between 2 and 7 amino acids, between 3 and 7 amino acids, or between 4 and 7 amino acids.

In a further embodiment of any one of said aspects, either one of X, or (B), can be a basic amino acid. In another embodiment, either one of X, or (B), is any amino acid except for C, S, or T. In yet a further embodiment, either one of X, or (B), is any amino acid except for a basic amino acid.

The peptides of the present invention have the advantage that cytolytic CD4+ T cells which have been generated using these peptides have an increased IFN-gamma and sFasL production compared to prior art peptides. Also Granzyme B production in said CD4+ T cells is believed to be increased.

The increased expression levels of these markers are indications of a greater capacity of the peptides of the present invention to generate cytolytic CD4+ T cells compared to the prior art peptides.

### BRIEF DESCRIPTION OF THE DRAWINGS

FIG. 1: represents kinetics of the reducing activities of immunogenic peptides P91 to P108. DTT is used as a positive control, while Blank represents the assay buffer. The results are expressed in Relative Fluorescent Units (RFU) over time. The assay is described in detail in the Examples section.

### DETAILED DESCRIPTION OF THE INVENTION

The present invention will be described with respect to particular embodiments but the invention is not limited thereto and is limited only by the appending claims. Any reference signs in the claims shall not be construed as limiting the scope. The following terms or definitions are provided solely to aid in the understanding of the invention. Unless specifically defined herein, all terms used herein have the same meaning as they would have to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 12 and 200 amino acids, connected by peptide bonds, but which can comprise non-amino acid structures.

The term “immunogenic peptide” as used herein refers to a peptide that is immunogenic, i.e. that comprises a T-cell epitope capable of eliciting an immune response.

Peptides according to the invention can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically a protein (with or without polysaccharides) or made of proteic composition comprising one or more hapten(s) and comprising T or NKT cell epitopes.

The term “antigenic protein” as used herein refers to a protein comprising one or more T or NKT cell epitopes. An auto-antigen or auto-antigenic protein as used herein refers to a human or animal protein or fragment thereof present in the body, which elicits an immune response within the same human or animal body.

The term “food or pharmaceutical antigenic protein” refers to an antigenic protein present in a food or pharmaceutical product, such as in a vaccine.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B-, or T-, or NKT cell, and which is able, by said binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T lymphocyte. The term encompasses both NKT cell epitopes and MHC class II T cell epitopes as defined herein. Whether an epitope is dominant, sub-dominant or minor depends on the immune reaction elicited against the epitope. Dominance depends on the frequency at which such epitopes are recognised by T cells and able to activate them, among all the possible T cell epitopes of a protein.

The identification and selection of a T-cell epitope from antigenic proteins is known to a person skilled in the art.

To identify an epitope suitable in the context of the present invention, isolated peptide sequences of an antigenic protein are tested by, for example, T cell biology techniques, to determine whether the peptide sequences elicit a T cell response. Those peptide sequences found to elicit a T cell response are defined as having T cell stimulating activity.

Human T cell stimulating activity can further be tested by culturing T cells obtained from e.g. an individual having T1D, with a peptide/epitope derived from the auto-antigen involved in T1D and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

Non-natural (or modified) T-cell epitopes can further optionally be tested on their binding affinity to MHC class II molecules or CD1d molecules. This can be performed in different ways. For instance, soluble HLA class II molecules are obtained by lysis of cells homozygous for a given class II or CD1d molecule. The latter is purified by affinity chromatography. Soluble class II molecules or CD1d are incubated with a biotin-labelled reference peptide produced according to its strong binding affinity for that class II molecule or CD1d molecule. Peptides to be assessed for class II binding or CD1d binding are then incubated at different concentrations and their capacity to displace the reference peptide from its class II binding is calculated by addition of neutravidin.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxy terminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol. 409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174, Guan et al. (2003) Appl. Bioinformatics 2, 63-66 (MHCPred) and Singh and Raghava (2001) Bioinformatics 17, 1236-1237 (Propred). More particularly, such algorithms allow the prediction within an antigenic protein of one or more octa- or nonapeptide sequences which will fit into the groove of an MHC II molecule and this for different HLA types or that bind to the CD1d molecule.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAS DQB1, HLA-DRA, and HLA-DRB1. In humans, the MHC is divided into three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2).

Class I MHC molecules are expressed on virtually all nucleated cells.

Peptide fragments presented in the context of class I MHC molecules are recognised by CD8+ T lymphocytes (cytolytic T lymphocytes or CTLs). CD8+ T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N- and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P−1, P−2 and so on, amino acids C-terminal of the epitope are numbered P+1, P+2 and so on. Peptides recognised by MHC class II molecules and not by MHC class I molecules are referred to as MHC class II restricted T cell epitopes.

The term “NKT cell epitope” refers to a part of an antigenic protein that is specifically recognized and bound by a receptor at the cell surface of an NKT cell. In particular, a NKT cell epitope is an epitope bound by CD1d molecules. The NKT cell epitope has a general motif [FWYHT]-X(2)-[VILM]-X(2)-[FWYHT] (SEQ ID NO: 127). Alternative versions of this general motif have at position 1 and/or position 7 the alternatives [FWYH], thus [FWYH]-X(2)-[VILM]-X(2)-[FWYH] (SEQ ID NO: 128).

Alternative versions of this general motif have at position 1 and/or position 7 the alternatives [FWYT], [FWYT]-X(2)-[VILM]-X(2)-[FWYT] (SEQ ID NO: 129). Alternative versions of this general motif have at position 1 and/or position 7 the alternatives [FWY], [FWY]-X(2)-[VILM]-X(2)-[FWY] (SEQ ID NO: 130).

Regardless of the amino acids at position 1 and/or 7, alternative versions of the general motif have at position 4 the alternatives [ILM], e.g. [FWYH]-X(2)-[ILM]-X(2)-[FWYH] (SEQ ID NO: 131) or [FWYHT]-X(2)-[ILM]-X(2)-[FWYHT] (SEQ ID NO: 132) or [FWY]-X(2)-[ILM]-X(2)-[FWY] (SEQ ID NO: 133).

A CD1d binding motif in a protein can be identified by scanning a sequence for the above sequence motifs, either by hand, either by using an algorithm such as ScanProsite De Castro E. et al. (2006) Nucleic Acids Res. 34 (Web Server issue):W362-W365.

“Natural killer T” or “NKT” cells constitute a distinct subset of non-conventional T lymphocytes that recognize antigens presented by the non-classical MHC complex molecule CD1d. Two subsets of NKT cells are presently described. Type I NKT cells, also called invariant NKT cells (INKT), are the most abundant. They are characterized by the presence of an alpha-beta T cell receptor (TCR) made of an invariant alpha chain, ValphaI4 in the mouse and Valpha24 in humans. This alpha chain is associated to a variable though limited number of beta chains. Type 2 NKT cells have an alpha-beta TCR but with a polymorphic alpha chain. However, it is apparent that other subsets of NKT cells exist, the phenotype of which is still incompletely defined, but which share the characteristics of being activated by glycolipids presented in the context of the CD1d molecule.

NKT cells typically express a combination of natural killer (NK) cell receptor, including NKG2D and NK1.1. NKT cells are part of the innate immune system, which can be distinguished from the adaptive immune system by the fact that they do not require expansion before acquiring full effector capacity. Most of their mediators are preformed and do not require transcription. NKT cells have been shown to be major participants in the immune response against intracellular pathogens and tumor rejection. Their role in the control of autoimmune diseases and of transplantation rejection is also advocated.

The recognition unit, the CD1d molecule, has a structure closely resembling that of the MHC class I molecule, including the presence of beta-2 microglobulin. It is characterized by a deep cleft bordered by two alpha chains and containing highly hydrophobic residues, which accepts lipid chains. The cleft is open at both extremities, allowing it to accommodate longer chains. The canonical ligand for CD1d is the synthetic alpha galactosylceramide (alpha GalCer). However, many natural alternative ligands have been described, including glyco- and phospholipids, the natural lipid sulfatide found in myelin, microbial phophoinositol mannoside and alpha-glucuronosylceramide. The present consensus in the art (Matsuda et al (2008), Curr. Opinion Immunol., 20 358-368; Godfrey et al (2010), Nature rev. Immunol 11, 197-206) is still that CD1d binds only ligands containing lipid chains, or in general a common structure made of a lipid tail which is buried into CD1d and a sugar residue head group that protrudes out of CD1d.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the redox motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment. Such polynucleotides or nucleic acids molecules may be readily prepared using an automated synthesizers and the well-known codon-amino acid relationship of the genetic code. Such polynucleotides or nucleic acids may be incorporated into expression vectors, including plasmids, which are adapted for the expression of the polynucleotide or nucleic acid and production of the polypeptide in a suitable host such as bacterium, e.g. Escherichia coli, yeast cell, human cell, animal cell or plant cell. For therapeutic means, polynucleotides encoding the immunogenic peptides disclosed herein can be part of an expression system, cassette, plasmid or vector system such as viral and non-viral expression systems. Viral vectors known for therapeutic purposes are adenoviruses, adeno-associated viruses (AAVs), lentiviruses, and retroviruses. Non-viral vectors can be used as well and non-limiting examples include: transposon-based vector systems such as those derived from Sleeping Beauty (SB) or PiggyBac (PB). Nucleic acids can also be delivered through other carriers such as but not limited to nanoparticles, cationic lipids, liposomes etc.

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “allergic diseases” or “allergic disorders” as used herein refer to diseases characterised by hypersensitivity reactions of the immune system to specific substances called allergens (such as pollen, stings, drugs, or food). Allergy is the ensemble of signs and symptoms observed whenever an atopic individual patient encounters an allergen to which he has been sensitised, which may result in the development of various diseases, in particular respiratory diseases and symptoms such as bronchial asthma. Various types of classifications exist and mostly allergic disorders have different names depending upon where in the mammalian body it occurs. “Hypersensitivity” is an undesirable (damaging, discomfort-producing and sometimes fatal) reaction produced in an individual upon exposure to an antigen to which it has become sensitised; “immediate hypersensitivity” depends of the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases.

An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “type 1 diabetes” (T1D) or “diabetes type 1” (also known as “type 1 diabetes mellitus” or “immune mediated diabetes” or formerly known as “juvenile onset diabetes” or “insulin dependent diabetes”) is an autoimmune disorder that typically develops in susceptible individuals during childhood. At the basis of T1D pathogenesis is the destruction of most insulin-producing pancreatic beta-cells by an autoimmune mechanism. In short, the organism loses the immune tolerance towards the pancreatic beta-cells in charge of insulin production and induces an immune response, mainly cell-mediated, associated to the production of autoantibodies, which leads to the self-destruction of beta-cells.

The term “demyelination” as used within the framework of demyelinating diseases or disorders herein refers to damaging and/or degradation of myelin sheaths that surround axons of neurons which has as a consequence the formation of lesions or plaques. Due to demyelination, the signal conduction along the affected nerves is impaired, and may cause neurological symptoms such as deficiencies in sensation, movement, cognition, and/or other neurological function. The concrete symptoms a patient suffering from a demyelinating disease will vary depending on the disease and disease progression state. These may include a blurred and/or double vision, ataxia, clonus, dysarthria, fatigue, clumsiness, hand paralysis, hemiparesis, genital anaesthesia, incoordination, paresthesias, ocular paralysis, impaired muscle coordination, muscle weakness, loss of sensation, impaired vision, neurological symptoms, unsteady way of walking (gait), spastic paraparesis, incontinence, hearing problems, speech problems, and others. Demyelinating diseases may be stratified into central nervous system demyelinating diseases and peripheral nervous system. Alternatively, demyelinating diseases may be classified according to the cause of demyelination: destruction of myelin (demyelinating myelinoclastic), or abnormal and degenerative myelin (dysmyelinating leukodystrophic). MS is considered in the art a demyelinating disorder of the central nervous system (Lubetzki and Stankoff. (2014). Handb Clin Neurol. 122, 89-99). Other specific but non-limiting examples of such demyelinating diseases and disorders include: neuromyelitis optica (NMO), acute inflammatory demyelinating polyneuropathy (AIDP), Chronic inflammatory demyelinating polyneuropathy (CIDP), acute transverse myelitis, progressive multifocal leukoencephalopathy (PML), acute disseminated encephalomyelitis (ADEM) or other hereditary demyelinating disorders.

The term “Multiple Sclerosis”, abbreviated herein and in the art as “MS”, indicates an autoimmune disorder affecting the central nervous system. MS is considered the most common non-traumatic disabling disease in young adults (Dobson and Giovannoni, (2019) Eur. J. Neurol. 26(1), 27-40), and the most common autoimmune disorder affecting the central nervous system (Berer and Krishnamoorthy (2014) FEBS Lett. 588(22), 4207-4213). MS may manifest itself in a subject by a large number of different symptoms ranging from physical over mental to psychiatric problems. Typical symptoms include blurred or double vision, muscle weakness, blindness in one eye, and difficulties in coordination and sensation. In most cases, MS may be viewed as a two-stage disease, with early inflammation responsible for relapsing-remitting disease and delayed neurodegeneration causing non-relapsing progression, i.e. secondary and primary progressive MS. Although progress is being made in the field, a conclusive underlying cause of the disease remains hitherto elusive and over 150 single nucleotide polymorphisms have been associated with MS susceptibility (International Multiple Sclerosis Genetics Consortium Nat Genet. (2013). 45(11):1353-60). Vitamin D deficiency, smoking, ultraviolet B (UVB) exposure, childhood obesity and infection by Epstein-Barr virus have been reported to contribute to disease development (Ascherio (2013) Expert Rev Neurother. 13(12 Suppl), 3-9).

Hence, MS can be regarded as a single disease existing within a spectrum extending from relapsing (wherein inflammation is the dominant feature) to progressive (neurodegeneration dominant). Therefore, it is evident that the term Multiple sclerosis as used herein encompasses any type of Multiple Sclerosis belonging to any kind of disease course classification. In particular the invention is envisaged to be a potent treatment strategy patient diagnosed with, or suspected of having clinically Isolated Syndrome (CIS), relapse-remitting MS (RRMS), secondary progressive MS (SPMS), primary progressive MS (PPMS), and even MS-suspected radiology isolated syndrome (RIS). While strictly not considered a disease course of MS, RIS is used to classify subjects showing abnormalities on the Magnetic Resonance Imaging (MRI) of brain and/or spinal cord that correspond to MS lesions and cannot be prima facie explained by other diagnoses. CIS is a first episode (by definition lasting for over 24 hours) of neurologic symptoms caused by inflammation and demyelination in the central nervous system. In accordance with RIS, CIS classified subjects may or may not continue to develop MS, with subjects showing MS-like lesions on a brain MRI more likely to develop MS. RRMS is the most common disease course of MS with 85% of subjects having MS being diagnosed with RRMS. RRMS diagnosed patients are a preferred group of patients in view of the current invention. RRMS is characterized by attacks of new or increasing neurologic symptoms, alternatively worded relapses or exacerbations. In RRMS, said relapses are followed by periods or partial or complete remission of the symptoms, and no disease progression is experienced and/or observed in these periods of remission. RRMS may be further classified as active RRMS (relapses and/or evidence of new MRI activity), non-active RRMS, worsening RRMS (increasing disability over a specified period of time after a relapse, or not worsening RRMS. A portion of RRMS diagnosed subject will progress to the SPMS disease course, which is characterized by a progressive worsening of neurologic function, i.e. an accumulation of disability, over time. SPMS subclassifications can be made such as active (relapses and/or new MRI activity), not active, progressive (disease worsening over time), or non-progressive SPMS. Finally, PPMS is an MS disease course characterized by worsening of neurologic function and hence an accumulation of disability from the onset of symptoms, without early relapse or remission. Further PPMS subgroups can be formed such as active PPMS (occasional relapse and/or new MRI activity), non-active PPMS, progressive PPMS (evidence of disease worsening over time, regardless of new MRI activity) and non-progressive PPMS. In general, MS disease courses are characterized by substantial intersubject variability in terms of relapse and remission periods, both in severity (in case of relapse) and duration.

Several disease modifying therapies are available for MS, and therefore the current invention may be used as alternative treatment strategy, or in combination with these existing therapies. Non-limiting examples of active pharmaceutical ingredients include interferon beta-1a, interferon beta-1b, glatiramer acetate, glatiramer acetate, peginterferon beta-1a, teriflunomide, fingolimod, cladribine, siponimod, dimethyl fumarate, diroximel fumarate, ozanimod, alemtuzumab, mitoxantrone, ocrelizumab, and natalizumab. Alternatively, the invention may be used in combination with a treatment or medication aiming to relapse management, such as but not limited to methylprednisolone, prednisone, and adrenocorticotropic hormone(s) (ACTH). Further, the invention may be used in combination with a therapy aiming to alleviate specific symptoms. Non-limiting examples include medications aiming to improve or avoid symptoms selected from the group consisting of: bladder problems, bowel dysfunction, depression, dizziness, vertigo, emotional changes, fatigue, itching, pain, sexual problems, spasticity, tremors, and walking difficulties.

MS is characterized by three intertwined hallmark characteristics: 1) lesion formation in the central nervous system, 2) inflammation, and 3) degradation of myelin sheaths of neurons. Despite traditionally being considered a demyelinating disease of the central nervous system and white matter, more recently reports have surfaced that demyelination of the cortical and deep gray matter may exceed white matter demyelination (Kutzelnigg et al. (2005). Brain. 128(11), 2705-2712). Two main hypotheses have been postulated as to how MS is caused at the molecular level. The commonly accepted “outside-in hypothesis” is based on the activation of peripheral autoreactive effector CD4+ T cells which migrate to the central nervous system and initiate the disease process. Once in the central nervous system, said T cells are locally reactivated by APCs and recruit additional T cells and macrophages to establish inflammatory lesions. Noteworthy, MS lesions have been shown to contain CD8+ T cells predominantly found at the lesion edges, and CD4+ T cells found more central in the lesions. These cells are thought to cause demyelination, oligodendrocyte destruction, and axonal damage, leading to neurologic dysfunction. Additionally, immune-modulatory networks are triggered to limit inflammation and to initiate repair, which results in at least partial remyelination reflected by clinical remission. Nonetheless, without adequate treatment, further attacks often lead to progression of the disease.

MS onset is believed to originate well before the first clinical symptoms are detected, as evidenced by the typical occurrence of apparent older and inactive lesions on the MRI of patients. Due to advances in the development of diagnostic methods, MS can now be detected even before a clinical manifestation of the disease (i.e. pre-symptomatic MS). In the context of the invention, “treatment of MS” and similar expressions envisage treatment of, and treatment strategies for, both symptomatic and pre-symptomatic MS. In particular, when the tolerogenic peptides and/or resulting cytolytic CD4+ T cells are used for treating a pre-symptomatic MS patient, the disease is halted at such an early stage that clinical manifestations may be partially, or even completely avoided. MS wherein the subject is not fully responsive to a treatment of interferon beta is also encompassed within the term “MS”.

The term “Neuromyelitis Optica” or “NMO” and “NMO Spectrum Disorder (NMOSD)”, also known as “Devic's disease”, refers to an autoimmune disorder in which white blood cells and antibodies primarily attack the optic nerves and the spinal cord, but may also attack the brain (reviewed in Wingerchuk 2006, Int MS J. 2006 May; 13(2):42-50). The damage to the optic nerves produces swelling and inflammation that cause pain and loss of vision; the damage to the spinal cord causes weakness or paralysis in the legs or arms, loss of sensation, and problems with bladder and bowel function. NMO is a relapsing-remitting disease. During a relapse, new damage to the optic nerves and/or spinal cord can lead to accumulating disability. Unlike MS, there is no progressive phase of this disease. Therefore, preventing attacks is critical to a good long-term outcome. In cases associated with anti-MOG antibodies, it is considered that anti-MOG antibodies may trigger an attack on the myelin sheath resulting in demyelination. The cause of NMO in the majority of cases is due to a specific attack on auto-antigens. Up to a third of subjects may be positive for auto-antibodies directed against a component of myelin called myelin oligodendrocyte glycoprotein (MOG). People with anti-MOG related NMO similarly have episodes of transverse myelitis and optic neuritis. Particularly envisaged within the framework of this application is NMO induced by MOG autoantigens and/or caused by anti-MOG antibodies. Many NMO patients develop auto-antibodies against Aquaporin-4 (AQP4).

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal. Alternatively, where the administration is through gene-therapy, the amount of naked DNA or viral vectors is adjusted to ensure the local production of the relevant dosage of the peptide of the invention, derivative or homologue thereof.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

In this context, it is realised that peptide fragments are generated from antigens, typically in the context of epitope scanning. By coincidence such peptides may comprise in their sequence a T cell epitope (an MHC class II epitope or a CD1d binding epitope) and in their proximity a sequence with the modified redox motif as defined herein. Alternatively there can be an amino acid sequence of at most 11 amino acids, at most 7 amino acids, at most 4 amino acids, at most 2 amino acids between said epitope and said oxidoreductase motif, or even 0 amino acids (in other words the epitope and oxidoreductase motif sequence are immediately adjacent to each other). In preferred embodiment, such naturally occurring peptides are disclaimed.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X or B, is used for a position where any amino acid is accepted herein. The symbol Z is used for a position where any amino acid which is not a basic amino acid such as R, K, or H, or which is not W, or A, is accepted herein. Alternative amino acids can be indicated by listing the acceptable amino acids for a given position, between square brackets (‘[ ]’). For example: [CST] stands for an amino acid selected from Cys, Ser or Thr. Amino acids which are excluded as alternatives can be indicated by listing them between curly brackets (‘{ }’). For example: {AM} stands for any amino acid except Ala and Met. The different elements in a motif are optionally separated from each other by a hyphen (-). To distinguish between the amino acids, those outside the oxidoreductase motif can be called external amino acids, those within the redox motif are called internal amino acids.

A peptide, comprising a T cell epitope, e.g. an MHC class II T-cell epitope or an NKT-cell epitope (or CD1d binding peptide epitope) and a modified peptide motif sequence, having reducing activity is capable of generating a population of antigen-specific cytolytic CD4+ T-cells, respectively cytolytic NKT-cells towards antigen-presenting cells.

Accordingly, in its broadest sense, the invention relates to peptides which comprise at least one T-cell epitope (MHC class II T-cell epitope or an NKT-cell epitope) of an antigen (self or non-self) with a potential to trigger an immune reaction, and a modified thioreductase sequence motif with a reducing activity on peptide disulfide bonds. The T cell epitope and the modified redox motif sequence may be immediately adjacent to each other in the peptide or optionally separated by one or more amino acids (so called linker sequence). Optionally the peptide additionally comprises an endosome targeting sequence and/or additional “flanking” sequences.

The peptides of the invention comprise a T-cell epitope of an antigen (self or non self) with a potential to trigger an immune reaction, and a modified redox motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzoli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The modified redox motif may be positioned at the amino-terminus side of the T-cell epitope or at the carboxy-terminus of the T-cell epitope.

Peptide fragments with reducing activity are encountered in thioreductases which are small disulfide reducing enzymes including glutaredoxins, nucleoredoxins, thioredoxins and other thiol/disulfide oxidoreductases (Holmgren (2000) Antioxid. Redox Signal. 2, 811-820; Jacquot et al. (2002) Biochem. Pharm. 64, 1065-1069). They are multifunctional, ubiquitous and found in many prokaryotes and eukaryotes. They are known to exert reducing activity for disulfide bonds on proteins (such as enzymes) through redox active cysteines within conserved active domain consensus sequences well-known from e.g. Fomenko et al. ((2003) Biochemistry 42, 11214-11225; Fomenko et al. (2002) Prot. Science 11, 2285-2296), in which X stands for any amino acid. and WO2008/017517 comprising a cysteine at position 1 and/or 4. Thus the motif is either CXX[CST] (SEQ ID NO: 2) or [CST]XXC (SEQ ID NO: 1). Such domains are also found in larger proteins such as protein disulfide isomerase (PDI) and phosphoinositide-specific phospholipase C. The present invention has redesigned said motifs in search for more potency and activity.

As explained in detail further on, the peptides of the present invention can be made by chemical synthesis, which allows the incorporation of non-natural amino acids. Accordingly, “C” in the above recited redox modified redox motifs represents either cysteine or another amino acid with a thiol group such as mercaptovaline, homocysteine or other natural or non-natural amino acids with a thiol function. In order to have reducing activity, the cysteines present in a modified redox motif should not occur as part of a cystine disulfide bridge. Nevertheless, a redox modified redox motif may comprise modified cysteines such as methylated cysteine, which is converted into cysteine with free thiol groups in vivo.

Peptides may further comprise modifications to increase stability or solubility, such as modification of the N-terminal NH₂ group or the C terminal COOH group (e.g. modification of the COOH into a CONH₂ group).

In the peptides of the present invention comprising a modified redox motif, the motif is located such that, when the epitope fits into the MHC groove or binds the CD1d receptor, the motif remains outside of the MHC or CD1d receptor binding groove. The modified redox motif is placed either immediately adjacent to the epitope sequence within the peptide [in other words a linker sequence of zero amino acids between motif and epitope], or is separated from the T cell epitope by a linker comprising an amino acid sequence of 7 amino acids or less. More particularly, the linker comprises 1, 2, 3, 4, 5, 6, or 7 amino acids. Specific embodiments are peptides with a 0, 1 or 2 amino acid linker between epitope sequence and modified redox motif sequence. Apart from a peptide linker, other organic compounds can be used as linker to link the parts of the peptide to each other (e.g. the modified redox motif sequence to the T cell epitope sequence).

The peptides of the present invention can further comprise additional short amino acid sequences N or C-terminally of the sequence comprising the T cell epitope and the modified redox motif. Such an amino acid sequence is generally referred to herein as a ‘flanking sequence’. A flanking sequence can be positioned between the epitope and an endosomal targeting sequence and/or between the modified redox motif and an endosomal targeting sequence. In certain peptides, not comprising an endosomal targeting sequence, a short amino acid sequence may be present N and/or C terminally of the modified redox motif and/or epitope sequence in the peptide. More particularly a flanking sequence is a sequence of between 1 and 7 amino acids, most particularly a sequence of 1, 2, 3, or 4 amino acids, most preferably of 2 amino acids. Particularly preferred flanking sequences are a single or double lysine residue (K or KK).

The modified redox motif may be located N-terminally from the epitope. Alternatively, the modified redox motif may be located C-terminally from the epitope.

In certain embodiments of the present invention, peptides are provided comprising one epitope sequence and a modified redox motif sequence. In further particular embodiments, the modified redox motif occurs several times (1, 2, 3, 4 or even more times) in the peptide, for example as repeats of the modified redox motif which can be spaced from each other by one or more amino acids or as repeats which are immediately adjacent to each other. Alternatively, one or more modified redox motifs are provided at both the N and the C terminus of the T cell epitope sequence.

Other variations envisaged for the peptides of the present invention include peptides which contain repeats of a T cell epitope sequence wherein each epitope sequence is preceded and/or followed by the modified redox motif (e.g. repeats of “modified redox motif-epitope” or repeats of “modified redox motif-epitope-modified redox motif’). Herein the modified redox motifs can all have the same sequence but this is not obligatory. It is noted that repetitive sequences of peptides which comprise an epitope which in itself comprises the modified redox motif will also result in a sequence comprising both the ‘epitope’ and a ‘modified redox motif’. In such peptides, the modified redox motif within one epitope sequence functions as a modified redox motif outside a second epitope sequence.

Typically the peptides of the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins. A T-cell epitope present within a peptide consist of between 7 and 30 amino acids, such as between 8 and 25 amino acids, yet more particularly of between 8 and 16 amino acids, yet most particularly consists of 8, 9, 10, 11, 12, 13, 14, 15 or 16 amino acids.

In a more particular embodiment, the T cell epitope consists of a sequence of 7, 8, or 9 amino acids. In a further particular embodiment, the T-cell epitope is an epitope, which is presented to T cells by MHC-class II molecules [MHC class II restricted T cell epitopes]. Typically T cell epitope sequence refers to the octapeptide or more specifically nonapeptide sequence which fits into the cleft of an MHC II protein.

In a more particular embodiment, the T cell epitope consists of a sequence of 7, 8, or 9 amino acids. In a further particular embodiment, the T-cell epitope is an epitope, which is presented by CD1d molecules [NKT cell epitopes]. Typically NKT cell epitope sequence refers to the 7 amino acid peptide sequence which binds to and is presented by the CD1d protein.

The T cell epitope of the peptides of the present invention can correspond either to a natural epitope sequence of a protein or can be a modified version thereof, provided the modified T cell epitope retains its ability to bind within the MHC cleft or to bind the CD1d receptor, similar to the natural T cell epitope sequence. The modified T cell epitope can have the same binding affinity for the MHC protein or the CD1d receptor as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC or CD1d complex compensates for the lowered affinity of the modified epitope for the MHC or CD1d molecule.

The sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and corresponds to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Alternatively, the present invention relates to the production of peptides containing hydrophobic residues that confer the capacity to bind to the CD1d molecule. Upon administration, such peptides are taken up by APC, directed to the late endosome where they are loaded onto CD1d and presented at the surface of the APC. Said hydrophobic peptides being characterized by a motif corresponding to the general sequence [FW]-XX-[ILM]-XX-[FWTH] (SEQ ID NO: 134) or [FWTH]-XX-[ILM]-XX-[FW] (SEQ ID NO: 135) in which positions P1 and P7 are occupied by hydrophobic residues such as phenylalanine (F) or tryptophan (W). P7 is however permissive in the sense that it accepts alternative hydrophobic residues to phenylalanine or tryptophan, such as threonine (T) or histidine (H). The P4 position is occupied by an aliphatic residue such as isoleucine (I), leucine (L) or methionine (M). The present invention relates to peptides made of hydrophobic residues which naturally constitute a CD1d binding motif. In some embodiment, amino acid residues of said motif are modified, usually by substitution with residues which increase the capacity to bind to 15 CD1d. In a specific embodiment, motifs are modified to fit more closely with the general motif [FW]-XX-[ILM]-XX-[FWTH] (SEQ ID NO: 134). More particularly, peptides are produced to contain a F or W at position 7.

Accordingly, the present invention envisages peptides of antigenic proteins and their use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides of the invention are generated by coupling a reducing compound, more particularly a reducing modified redox motif as described herein, N-terminally or C-terminally to a T cell epitope of the antigenic protein (either directly adjacent thereto or with a linker of at most 10, more particularly at most 7 amino acids). Moreover the T cell epitope sequence of the protein and/or the modified redox motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or ‘naturally occurring’.

The peptides of the present invention can vary substantially in length. The length of the peptides can vary from 13 or 14 amino acids, i.e. consisting of an epitope of 8-9 amino acids, adjacent thereto the modified redox motif 5 amino acids with the histidine, up to 20, 25, 30, 40 or 50 amino acids. For example, a peptide may comprise an endosomal targeting sequence of 40 amino acids, a flanking sequence of about 2 amino acids, a motif as described herein of 5 amino acids, a linker of 4 amino acids and a T cell epitope peptide of 9 amino acids.

Accordingly, in particular embodiments, the complete peptide consists of between 13 amino acids up 20, 25, 30, 40, 50, 75 or 100 amino acids. More particularly, where the reducing compound is a modified redox motif as described herein, the length of the (artificial or natural) sequence comprising the epitope and modified redox motif optionally connected by a linker (referred to herein as ‘epitope-modified redox motif’ sequence), without the endosomal targeting sequence, is critical. The ‘epitope-modified redox motif’ more particularly has a length of 13, 14, 15, 16, 17, 18 or 19 amino acids. Such peptides of 13 or 14 to 19 amino acids can optionally be coupled to an endosomal targeting signal of which the size is less critical.

As detailed above, in particular embodiments, the peptides of the present invention comprise a reducing modified redox motif as described herein linked to a T cell epitope sequence.

In further particular embodiments, the peptides of the invention are peptides comprising T cell epitopes which do not comprise an amino acid sequence with redox properties within their natural sequence.

However, in alternative embodiments, the T cell epitope may comprise any sequence of amino acids ensuring the binding of the epitope to the MHC cleft or to the CD1d molecule. Where an epitope of interest of an antigenic protein comprises a modified redox motif such as described herein within its epitope sequence, the immunogenic peptides according to the present invention comprise the sequence of a modified redox motif as described herein and/or of another reducing sequence coupled N- or C-terminally to the epitope sequence such that (contrary to the modified redox motif present within the epitope, which is buried within the cleft) the attached modified redox motif can ensure the reducing activity.

Accordingly the T cell epitope and motif are immediately adjacent or separated from each other and do not overlap. To assess the concept of “immediately adjacent” or “separated”, the 8 or 9 amino acid sequence which fits in the MHC cleft or CD1d molecule is determined and the distance between this octapeptide or nonapeptide with the redox motif tetrapeptide or modified redox motif pentapeptide including histidine is determined.

Generally, the peptides of the present invention are not natural (thus no fragments of proteins as such) but artificial peptides which contain, in addition to a T cell epitope, a modified redox motif as described herein, whereby the modified redox motif is immediately separated from the T cell epitope by a linker consisting of up to seven, most particularly up to four or up to 2 amino acids.

It has been shown that upon administration (i.e. injection) to a mammal of a peptide comprising a oxidoreductase motif and an MHC class II T-cell epitope (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells.

Additionally, it has been shown that upon administration (i.e. injection) to a mammal of a peptide comprising a oxidoreductase motif and an NKT-cell epitope (or a composition comprising such a peptide), the peptide elicits the activation of T cells recognising the antigen derived T cell epitope and provides an additional signal to the T cell through binding to the CD1d surface receptor. This activation results in NKT cells acquiring cytolytic properties for the cell presenting the T cell epitope. In this way, the peptides or composition comprising the peptides described in the present invention, which contain an antigen-derived T cell epitope and, outside the epitope, a modified redox motif can be used for direct immunisation of mammals, including human beings. The invention thus provides peptides of the invention or derivatives thereof, for use as a medicine. Accordingly, the present invention provides therapeutic methods which comprise administering one or more peptides according to the present invention to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class II presentation or CD1d receptor binding, elicit cytolytic CD4+ T-cells or NKT cells respectively.

The immunogenic properties of the peptides of the present invention are of particular interest in the treatment and prevention of immune reactions.

Peptides described herein are used as medicament, more particularly used for the manufacture of a medicament for the prevention or treatment of an immune disorder in a mammal, more in particular in a human.

The present invention describes methods of treatment or prevention of an immune disorder of a mammal in need for such treatment or prevention, by using the peptides of the invention, homologues or derivatives thereof, the methods comprising the step of administering to said mammal suffering or at risk of an immune disorder a therapeutically effective amount of the peptides of the invention, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases.

The peptides of the invention or the pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide according to the invention or the pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 μg or between 850 and 1300 μg, depending on the condition of the patient and severity of disease. Dosage regimen can comprise the administration in a single dose or in 2, 3, 4, 5, or more doses, either simultaneously or consecutively. Exemplary non-limiting administration schemes are the following:
A low dose scheme comprising the SC administration of 50 μg of peptide in two separate injections of 25 μg each (100 μL each) followed by three consecutive injections of 25 μg of peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of peptide as two separate injections of 37.5 μg each (150 μL each).
A high dose scheme comprising the SC administration of 450 μg of peptide in two separate injections of 225 μg each (900 μL each) followed by three consecutive administrations of 225 μg of peptide as two separate injections of 112.5 μg each (450 μL each).

An exemplary dose scheme of an immunogenic peptide comprising a known oxidoreductase motif and a T-cell epitope can be found on ClinicalTrials.gov under Identifier NCT03272269.

The present invention provides for immunogenic peptides comprising an improved oxidoreductase motif and a T-cell epitope of an antigenic protein, optionally separated by a linker of between 0 and 7 amino acids.

The terms “oxidoreductase motif”, “thiol-oxidoreductase motif”, “thioreductase motif”, “thioredox motif” or “redox motif” are used herein as synonymous terms and refers to motifs involved in the transfer of electrons from one molecule (the reductant, also called the hydrogen or electron donor) to another (the oxidant, also called the hydrogen or electron acceptor).

In preferred embodiments, said oxidoreductase motif is selected from the group comprising the following general amino acids sequence:

(SEQ ID NOs: 96-109)
Z(B)ₙ[CST]XₘC-
or
(SEQ ID NOs: 110-123)
Z(B)ₙCXₘ[CST]-

wherein Z is any amino acid or non-natural amino acid, preferably excluding basic amino acids such as; R (Arginine), K (Lysine) and H (Histidine), and preferably excluding amino acids D (Aspartate), E (Glutamate), and/or A (Alanine);
wherein (B) is any amino acid;
wherein n is an integer of 0 to 2;
wherein X is any amino acid;
wherein m is an integer of 0 to 4, preferably wherein m is 1, 2, or 3, more preferably wherein m is 2; wherein the hyphen (-) in said oxidoreductase motif indicates the point of attachment of the oxidoreductase motif to the N-terminal end of the linker (c) or the epitope (b), or to the C-terminal end of the linker (c) or the epitope (b).

Preferably said Z is selected from the group consisting of: W, G, S, T, C, V, L, I, M, P, F, Y, N, and Q, or is any non-natural non-basic amino acid.

Preferably said X is selected from the group consisting of: G, A, V, L, I, M, F, W, P, S, T, C, Y, N, Q, D, and E.

In further particular embodiments of said peptide, Z(B)ₙ is selected from: W, P, G, KW, KP, KG, RW, RP, RG, HW, HP, HG, PH, WH, GH, PK, WK, GK, PR, WR, and GR. In preferred embodiments, X is any amino acid except for C, S, or T.

In preferred embodiments, X is any amino acid except for a basic amino acid or non-natural basic amino acid as defined herein.

In preferred embodiment, the immunogenic peptide can contain a flanking amino acid sequence of between 1 and 7 amino acids, most particularly a sequence of 1, 2, 3, or 4 amino acids, most preferably of 2 amino acids. In particularly preferred embodiments, said flanker sequence comprises or consists of one or two or more K residues (lysine amino acid residues).

In a preferred embodiment, said oxidoreductase motif is Z(B)ₙ[CST]PYC (SEQ ID NOs: 136-138) or Z(B)ₙCPY[CST] (SEQ ID NOs: 139-141), such as Z(B)ₙCPYC (SEQ ID NOs: 142-144), Z(B)ₙSPYC (SEQ ID NOs: 145-147), Z(B)ₙTPYC (SEQ ID NOs: 148-150), Z(B)ₙCPYC (SEQ ID NOs: 151-153), Z(B)ₙCPYS (SEQ ID NOs: 154-156), or Z(B)ₙCPYT (SEQ ID NOs: 157-159). In any one of these motifs, Z can be any amino acid, preferably not a basic amino acid such as R, K and H, and optionally also excluding D, E, and/or A. In any one of these motifs, (B) can be any amino acid, preferably not a basic amino acid, and n is an integer from 0 to 2. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

In a preferred embodiment, said oxidoreductase motif is Z(B)ₙ[CST]HGC (SEQ ID NOs: 160-162) or Z(B)ₙCHG[CST] (SEQ ID NOs: 163-165), such as Z(B)ₙCHGC (SEQ ID NOs: 166-168), Z(B)ₙSHGC (SEQ ID NOs: 169-171), Z(B)ₙTHGC (SEQ ID NOs: 172-174), Z(B)ₙCHGC (SEQ ID NOs: 175-177), Z(B)ₙCHGS (SEQ ID NOs: 178-180), or Z(B)ₙCHGT (SEQ ID NOs: 181-183). In any one of these motifs, Z can be any amino acid, preferably not a basic amino acid such as R, K and H, and optionally also excluding D, E, and/or A. In any one of these motifs, (B) can be any amino acid, preferably not a basic amino acid, and n is an integer from 0 to 2. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

In a preferred embodiment, said oxidoreductase motif is Z(B)ₙ[CST]GPC (SEQ ID NOs: 184-186) or Z(B)ₙCGP[CST] (SEQ ID NOs: 187-189), such as Z(B)ₙCGPC (SEQ ID NOs: 190-192), Z(B)ₙSGPC (SEQ ID NOs: 193-195), Z(B)ₙTGPC (SEQ ID NOs: 196-198), Z(B)ₙCGPC (SEQ ID NOs: 199-201), Z(B)ₙCGPS (SEQ ID NOs: 202-204), or Z(B)ₙCGPT (SEQ ID NOs: 205-207). In any one of these motifs, Z can be any amino acid, preferably not a basic amino acid such as R, K and H and optionally also excluding D, E, and/or A. In specific examples, Z is K, R or a non-natural amino acid as defined herein. In any one of these motifs, (B) can be any amino acid, preferably not a basic amino acid, and n is an integer from 0 to 2. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

In a preferred embodiment, said oxidoreductase motif is Z(B)ₙ[CST]GHC (SEQ ID NOs: 208-210) or Z(B)ₙCGH[CST] (SEQ ID NOs: 211-213), such as Z(B)ₙCGHC (SEQ ID NOs: 214-216), Z(B)ₙSGHC (SEQ ID NOs: 217-219), Z(B)ₙTGHC (SEQ ID NOs: 220-222), Z(B)ₙCGHC (SEQ ID NOs: 223-225), Z(B)ₙCGHS (SEQ ID NOs: 226-228), or Z(B)ₙCGHT (SEQ ID NOs: 229-231). In any one of these motifs, Z can be any amino acid, preferably not a basic amino acid such as R, K and H, and optionally also excluding D, E, and/or A. In any one of these motifs, (B) can be any amino acid, preferably not a basic amino acid, and n is an integer from 0 to 2. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

In a preferred embodiment, said oxidoreductase motif is Z(B)ₙ[CST]GFC (SEQ ID NOs: 232-234) or Z(B)ₙCGF[CST] (SEQ ID NOs: 235-237), such as Z(B)ₙCGFC (SEQ ID NOs: 238-240), Z(B)ₙSGFC (SEQ ID NOs: 241-243), Z(B)ₙTGFC (SEQ ID NOs: 244-246), Z(B)ₙCGFC (SEQ ID NOs: 247-249), Z(B)ₙCGFS (SEQ ID NOs: 250-252), or Z(B)ₙCGFT (SEQ ID NOs: 253-255). In any one of these motifs, Z can be any amino acid, preferably not a basic amino acid such as R, K and H, and optionally also excluding D, E, and/or A. In any one of these motifs, (B) can be any amino acid, preferably not a basic amino acid, and n is an integer from 0 to 2. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

In a preferred embodiment, said oxidoreductase motif is Z(B)ₙ[CST]RLC (SEQ ID NOs: 256-258) or Z(B)ₙCRL[CST] (SEQ ID NOs: 259-261), such as Z(B)ₙCRLC (SEQ ID NOs: 262-264), Z(B)ₙSRLC (SEQ ID NOs: 265-267), Z(B)ₙTRLC (SEQ ID NOs: 268-270), Z(B)ₙCRLC (SEQ ID NOs: 271-273), Z(B)ₙCRLS (SEQ ID NOs: 274-276), or Z(B)ₙCRLT (SEQ ID NOs: 277-279). In any one of these motifs, Z can be any amino acid, preferably not a basic amino acid such as R, K and H, and optionally also excluding D, E, and/or A. In any one of these motifs, (B) can be any amino acid, preferably not a basic amino acid, and n is an integer from 0 to 2. In preferred embodiments of said peptide, Z(B)ₙ does not have the following sequence: K; KH; R; or RH. In further particular embodiments of said peptide, Z(B)ₙ is selected from: W, P, KW, KP, RW, RP, HW, HP, PH, WH, PK, WK, PR, and WR.

In a preferred embodiment, said oxidoreductase motif is Z(B)ₙ[CST]HPC (SEQ ID NOs: 280-282) or Z(B)ₙCHP[CST] (SEQ ID NOs: 283-285), such as Z(B)ₙCHPC (SEQ ID NOs: 286-288), Z(B)ₙSHPC (SEQ ID NOs: 289-291), Z(B)ₙTHPC (SEQ ID NOs: 292-294), Z(B)ₙCHPC (SEQ ID NOs: 295-297), Z(B)ₙCHPS (SEQ ID NOs: 298-300), or Z(B)ₙCHPT (SEQ ID NOs: 301-303). In any one of these motifs, Z can be any amino acid, preferably not a basic amino acid such as R, K and H, and optionally also excluding D, E, and/or A. In any one of these motifs, (B) can be any amino acid, preferably not a basic amino acid, and n is an integer from 0 to 2. In preferred embodiments of said peptide, Z(B)ₙ has the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Specific examples of immunogenic peptides according to the invention are the following: Z(B)ₙ-CPYC-V-QYIKANSKFIGIT-EL (SEQ ID NOs: 304-306), wherein Z(B)ₙ is as defined herein, wherein -CPYC- (SEQ ID NO: 307) represents the thioredox motif, wherein -V- is the linker, wherein -QYIKANSKFIGIT- (SEQ ID NO: 308) is a T-cell epitope of tetanus toxin and wherein -EL is a C-terminal flanking sequence. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Z(B)ₙ-CHGC-V-QYIKANSKFIGIT-EL (SEQ ID NOs: 309-311), wherein Z(B)ₙ is as defined herein, wherein -CHGC- (SEQ ID NO: 312) represents the thioredox motif, wherein -V- is the linker, wherein -QYIKANSKFIGIT- (SEQ ID NO: 308) is a T-cell epitope of tetanus toxin and wherein -EL is a C-terminal flanking sequence. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Z(B)ₙ-CPYC-GG-FIGLMYY (SEQ ID NOs: 313-315), wherein Z(B)ₙ is as defined herein, wherein -CPYC- (SEQ ID NOs: 307) represents the thioredox motif, wherein -GG- is the linker, wherein -FIGLMYY- (SEQ ID NOs: 316) is an NKT-cell epitope of a hexon protein of adenovirus (Ad5) and wherein there is no C-terminal flanking sequence. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Z(B)ₙ-CPYC-GW-YRSPFSRVV-HLYR (SEQ ID NOs: 317-319), wherein Z(B)ₙ is as defined herein, wherein -CPYC- (SEQ ID NO: 307) represents the thioredox motif, wherein -GW- is the linker, wherein -YRSPFSRVV- (SEQ ID NO: 320) is an T-cell epitope of the MOG protein and wherein -HLYR (SEQ ID NO: 321) is a C-terminal flanking sequence. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Z(B)ₙ-CPYC-GW-YRSPFSRVV-K (SEQ ID NOs: 322-324) or Z(B)ₙ-CPYC-GW-YRSPFSRVV-KK (SEQ ID NOs: 325-327) wherein Z(B)ₙ is as defined herein, wherein -CPYC- (SEQ ID NO: 307) represents the thioredox motif, wherein -GW- is the linker, wherein -YRSPFSRVV- (SEQ ID NO: 320) is an T-cell epitope of the MOG protein and wherein -K or -KK is a C-terminal flanking sequence. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

Z(B)ₙ-CPYC-VRY-FLRVPSWKI-TLF (SEQ ID NOs: 328-330), wherein Z(B)ₙ is as defined herein, wherein -CPYC- (SEQ ID NO: 307) represents the thioredox motif, wherein -VRY- is the linker, wherein -FLRVPSWKI- (SEQ ID NO: 331) is an T-cell epitope of the MOG protein and wherein -TLF is a C-terminal flanking sequence. In preferred embodiments of said peptide, Z(B)ₙ does not have the following sequence: K; KH; R; or RH. In further particular embodiments of said peptide, Z(B)ₙ is selected from: W, P, G, KW, KP, KG, RW, RP, RG, HW, HP, HG, PH, WH, GH, PK, WK, GK, PR, WR, and GR. The C-terminal flanker sequence TLF can be supplemented by one or two K residues.

Z(B)ₙ-CPYC-SLQP-LALEGSLQK-RG (SEQ ID NOs: 332-334), wherein Z(B)ₙ is as defined herein, wherein -CPYC- (SEQ ID NO: 307) represents the thioredox motif, wherein -SLQP- (SEQ ID NO: 335) is the linker, wherein -LALEGSLOK- (SEQ ID NO: 336) is an MHC class-II T-cell epitope of the (pro)insulin protein and wherein -RG is a C-terminal flanking sequence. In preferred embodiments of said peptide, Z(B)ₙ does not have the following sequence: K; KH; R; or RH.

In a particularly preferred embodiment, Z is W or P and n is 0, such as in sequences: W-CPYC-SLQP-LALEGSLOK-RG (SEQ ID NO: 337) and P-CPYC-SLQP-LALEGSLQK-RG (SEQ ID NO: 338). In further particular embodiments of said peptide, Z(B)ₙ is selected from: W, P, G, KW, KP, KG, RW, RP, RG, HW, HP, HG, PH, WH, GH, PK, WK, GK, PR, WR, and GR.

The peptides of the present invention can also be used in diagnostic in vitro methods for detecting class II restricted CD4+T cells in a sample. In this method a sample is contacted with a complex of an MHC class II molecule and a peptide according to the present invention. The CD4+ T cells are detected by measuring the binding of the complex with cells in the sample, wherein the binding of the complex to a cell is indicative for the presence of CD4+T cells in the sample. The complex can be a fusion protein of the peptide and an MHC class II molecule. Alternatively MHC molecules in the complex are tetramers. The complex can be provided as a soluble molecule or can be attached to a carrier.

The peptides of the present invention can also be used in diagnostic in vitro methods for detecting NKT cells in a sample. In this method a sample is contacted with a complex of a CD1d molecule and a peptide according to the present invention. The NKT cells are detected by measuring the binding of the complex with cells in the sample, wherein the binding of the complex to a cell is indicative for the presence of NKT cells in the sample. The complex can be a fusion protein of the peptide and a CD1d molecule.

Accordingly, in particular embodiments, the methods of treatment and prevention of the present invention comprise the administration of an immunogenic peptide as described herein, wherein the peptide comprise a T cell epitope of an antigenic protein which plays a role in the disease to be treated (for instance such as those described above). In further particular embodiments, the epitope used is a dominant epitope.

Peptides in accordance of the present invention will be prepared by synthesising a peptide wherein T cell epitope and modified redox motif will be separated by 0 to 5 amino acids. In certain embodiments the modified redox motif can be obtained by introducing 1, 2 or 3 mutations outside the epitope sequence, to preserve the sequence context as occurring in the protein. Typically amino-acids in P−2 and P−1, as well as in P+10 and P+11, with reference to the nonapeptide which are part of the natural sequence are preserved in the peptide sequence. These flanking residues generally stabilize the binding to MHC class II or CD1d molecules. In other embodiments the sequence N terminal or C terminal of the epitope will be unrelated to the sequence of the antigenic protein containing the T cell epitope sequence.

Thus based upon the above methods for designing a peptide, a peptide is generated by chemical peptide synthesis, recombinant expression methods or in more exceptional cases, proteolytic or chemical fragmentation of proteins.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T or NKT cells which are cytolytic via an apoptotic pathway for antigen presenting cells presenting the antigen which contains the epitope sequence which is also present in the peptide with the modified redox motif.

The peptides of the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains such as methylated cysteine.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992) Int. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically this is optimised by adjusting the sequence of the peptide. Optionally, the peptide can be modified after synthesis (chemical modifications e.g. adding/deleting functional groups) using techniques known in the art.

The mechanism of action of immunogenic peptides comprising a standard oxidoreductase motif and an MHC class II T-cell epitope is substantiated with experimental data disclosed in the above cited PCT application WO2008/017517 and publications of the present inventors. The mechanism of action of immunogenic peptides comprising a standard oxidoreductase motif and a CD1d binding NKT-cell epitope is substantiated with experimental data disclosed in the above cited PCT application WO2012/069568 and publications of the present inventors.

The present invention provides methods for generating antigen-specific cytolytic CD4+ T-cells (when using an immunogenic peptide as disclosed herein comprising an MHC class II epitope), or antigen-specific cytolytic NKT-cells (when using an immunogenic peptide as disclosed herein comprising an NKT cell epitope binding the CD1d molecule) either in vivo or in vitro.

The present invention describes in vivo methods for the production of the antigen-specific CD4+ T cells or NKT cells. A particular embodiment relates to the method for producing or isolating the CD4+ T cells or NKT cells by immunising animals (including humans) with the peptides of the invention as described herein and then isolating the CD4+ T cells or NKT cells from the immunised animals. The present invention describes in vitro methods for the production of antigen specific cytolytic CD4+ T cells or NKT cells towards APC. The present invention provides methods for generating antigen specific cytolytic CD4+T cells and NKT cells towards APC.

In one embodiment, methods are provided which comprise the isolation of peripheral blood cells, the stimulation of the cell population in vitro by an immunogenic peptide according to the invention and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4+T cells towards APC, obtainable by the methods of the present invention are of particular interest for the administration to mammals for immunotherapy, in the prevention of allergic reactions and the treatment of auto-immune diseases. Both the use of allogenic and autogeneic cells are envisaged.

Cytolytic CD4+ T cells populations are obtained as described herein below.

In one embodiment, the invention provides ways to expand specific NKT cells, with as a consequence increased activity comprising, but not limited to:
(i) increased cytokine production
(ii) increased contact- and soluble factor-dependent elimination of antigen-presenting cells. The result is therefore a more efficient response towards intracellular pathogens, autoantigens, allofactors, allergens, tumor cells and more efficient suppression of immune responses against graft and viral proteins used in gene therapy/gene vaccination.

The present invention also relates to the identification of NKT cells with required properties in body fluids or organs. The method comprises identification of NKT cells by virtue of their surface phenotype, including expression of NK1.1, CD4, NKG2D and CD244. Cells are then contacted with NKT cell epitopes defined as peptides able to be presented by the CD1d molecule. Cells are then expanded in vitro in the presence of IL-2 or IL-15 or IL-7.

Antigen-specific cytolytic CD4+ T cells or NKT cells as described herein can be used as a medicament, more particularly for use in adoptive cell therapy, more particularly in the treatment of acute allergic reactions and relapses of autoimmune diseases such as multiple sclerosis. Isolated cytolytic CD4+ T cells or NKT cells or cell populations, more particularly antigen-specific cytolytic CD4+ T cell or NKT cell populations generated as described are used for the manufacture of a medicament for the prevention or treatment of immune disorders. Methods of treatment by using the isolated or generated cytolytic CD4+ T cells or NKT cells are disclosed.

As explained in WO2008/017517 cytolytic CD4+ T cells towards APC can be distinguished from natural Treg cells based on expression characteristics of the cells. More particularly, a cytolytic CD4+T cell population demonstrates one or more of the following characteristics compared to a natural Treg cell population:

an increased expression of surface markers including CD103, CTLA-4, Fasl and ICOS upon activation, intermediate expression of CD25, expression of CD4, ICOS, CTLA-4, GITR and low or no expression of CD127 (IL7-R), no expression of CD27, expression of transcription factor T-bet and egr-2 (Krox-20) but not of the transcription repressor Foxp3, a high production of IFN-gamma and no or only trace amounts of IL-10, IL-4, IL-5, IL-13 or TGF-beta.

Further the cytolytic T cells express CD45RO and/or CD45RA, do not express CCR7, CD27 and present high levels of granzyme B and other granzymes as well as Fas ligand.

As explained in WO2008/017517 cytolytic NKT cells against towards APC can be distinguished from non-cytolytic NKT cells based on expression characteristics of the cells. More particularly, a cytolytic CD4+NKT cell population demonstrates one or more of the following characteristics compared to a non-cytolytic NKT cell population: expression of NK1.I, CD4, NKG2D and CD244. The peptides of the invention will, upon administration to a living animal, typically a human being, elicit specific T cells exerting a suppressive activity on bystander T cells.

In specific embodiments the cytolytic cell populations of the present invention are characterised by the expression of FasL and/or Interferon gamma. In specific embodiments the cytolytic cell populations of the present invention are further characterised by the expression of GranzymeB.

This mechanism also implies and the experimental results show that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules or CD1d molecules in the vicinity of T cells activated by peptides of the invention.

Isolated cell populations of the cell type having the characteristics described above, which, in addition are antigen-specific, i.e. capable of suppressing an antigen-specific immune response are disclosed.

The present invention provides pharmaceutical compositions comprising one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders. The pharmaceutical composition could for example be a vaccine suitable for treating or preventing immune disorders, especially airborne and foodborne allergy, as well as diseases of allergic origin. As an example described further herein of a pharmaceutical composition, a peptide according to the invention is adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class II presentation and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers. The present invention relates to pharmaceutical compositions, comprising, as an active ingredient, one or more peptides according to the invention, in admixture with a pharmaceutically acceptable carrier. The pharmaceutical composition of the present invention should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The term “pharmaceutically acceptable carrier” as used herein means any material or substance with which the active ingredient is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders. Suitable pharmaceutical carriers for use in the pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalenesulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanylphosphatidylcholine, dipalmitoylphoshatidylcholine and their mixtures.

Suitable non-ionic surfactants include polyethoxylated and poly propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “Mccutcheon's Detergents and Emulsifiers Annual” (MC Publishing Crop., Ridgewood, New Jersey, 1981), “Tensid-Taschenbuch”, 2nd ed. (Hanser Verlag, Vienna, 1981) and “Encyclopedia of Surfactants, (Chemical Publishing Co., New York, 1981). Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition.

Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Cytolytic CD4+T cells as obtained in the present invention, induce APC apoptosis after MHC-class II dependent cognate activation, affecting both dendritic and B cells, as demonstrated in vitro and in vivo, and (2) suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

The immunogenic peptides of the invention containing hydrophobic residues that confer the capacity to bind to the CD1d molecule. Upon administration, are taken up by APC, directed to the late endosome where they are loaded onto CD1d and presented at the surface of the APC. Once presented by CD1d molecule, the thioreductase motif in the peptides enhances the capacity to activate NKT cells, becoming cytolytic NKT cells. Said immunogenic peptides activate the production of cytokine, such as IFN-gamma, which will activate other effector cells including CD4+ T cells and CD8+ T cells. Both CD4+ and CD8+ T cells can participate in the elimination of the cell presenting the antigen as discussed in detail in WO2012/069568.

The present invention will now be illustrated by means of the following examples which are provided without any limiting intention. Furthermore, all references described herein are explicitly included herein by reference.

### EXAMPLES

### Example 1: Peptide Design

In order to assess the effect of additional flanking amino acids on the activity of the oxidoreductase motif in connection to a T-cell epitope, the following peptides (Tables 1 to 6) were synthesised and compared to an immunogenic peptide comprising anoxidoreductase motif not flanked by one or more amino acids or flanked by H. All peptides tested comprise a linker, a T-cell epitope and optionally a C-terminal flanking sequence.

TABLE 1
Immunogenic peptides with different oxidoreductase motifs based on the
CPYC motif and a Tetanus toxin MHC class II T cell epitope.

| Peptide- | Oxidoreductase | | | | | |
|---|---|---|---|---|---|---|
| Noº | Motif | | Linker | T-Epitope | C-term | SEQ ID NO |
| 1 | — | CPYC | V | QYIKANSKFIGIT | EL | 339 |
| 2 | D | CPYC | V | QYIKANSKFIGIT | EL | 340 |
| 3 | E | CPYC | V | QYIKANSKFIGIT | EL | 341 |
| 4 | S | CPYC | V | QYIKANSKFIGIT | EL | 342 |
| 5 | T | CPYC | V | QYIKANSKFIGIT | EL | 343 |
| 6 | N | CPYC | V | QYIKANSKFIGIT | EL | 344 |
| 7 | Q | CPYC | V | QYIKANSKFIGIT | EL | 345 |
| 8 | C | CPYC | V | QYIKANSKFIGIT | EL | 346 |
| 9 | G | CPYC | V | QYIKANSKFIGIT | EL | 347 |
| 10 | P | CPYC | V | QYIKANSKFIGIT | EL | 348 |
| 11 | A | CPYC | V | QYIKANSKFIGIT | EL | 349 |
| 12 | I | CPYC | V | QYIKANSKFIGIT | EL | 350 |
| 13 | L | CPYC | V | QYIKANSKFIGIT | EL | 351 |
| 14 | M | CPYC | V | QYIKANSKFIGIT | EL | 352 |
| 15 | F | CPYC | V | QYIKANSKFIGIT | EL | 353 |
| 16 | W | CPYC | V | QYIKANSKFIGIT | EL | 354 |
| 17 | Y | CPYC | V | QYIKANSKFIGIT | EL | 355 |
| 18 | V | CPYC | V | QYIKANSKFIGIT | EL | 356 |

TABLE 2
Immunogenic peptides with different oxidoreductase motifs based on the
CHGC motif and a Tetanus toxin MHC class II T cell epitope.

| Peptide- | Oxidoreductase | | | | | |
|---|---|---|---|---|---|---|
| Noº | Motif | | Linker | T-Epitope | C-term | SEQ ID NO |
| 19 | — | CHGC | V | QYIKANSKFIGIT | EL | 357 |
| 20 | D | CHGC | V | QYIKANSKFIGIT | EL | 358 |
| 21 | E | CHGC | V | QYIKANSKFIGIT | EL | 359 |
| 22 | S | CHGC | V | QYIKANSKFIGIT | EL | 360 |
| 23 | T | CHGC | V | QYIKANSKFIGIT | EL | 361 |
| 24 | N | CHGC | V | QYIKANSKFIGIT | EL | 362 |
| 25 | Q | CHGC | V | QYIKANSKFIGIT | EL | 363 |
| 26 | C | CHGC | V | QYIKANSKFIGIT | EL | 364 |
| 27 | G | CHGC | V | QYIKANSKFIGIT | EL | 365 |
| 28 | P | CHGC | V | QYIKANSKFIGIT | EL | 366 |
| 29 | A | CHGC | V | QYIKANSKFIGIT | EL | 367 |
| 30 | I | CHGC | V | QYIKANSKFIGIT | EL | 368 |
| 31 | L | CHGC | V | QYIKANSKFIGIT | EL | 369 |
| 32 | M | CHGC | V | QYIKANSKFIGIT | EL | 370 |
| 33 | F | CHGC | V | QYIKANSKFIGIT | EL | 371 |
| 34 | W | CHGC | V | QYIKANSKFIGIT | EL | 372 |
| 35 | Y | CHGC | V | QYIKANSKFIGIT | EL | 373 |
| 36 | V | CHGC | V | QYIKANSKFIGIT | EL | 374 |

TABLE 3
Immunogenic peptides with different oxidoreductase motifs based on
the CPYC motif and a hexon protein of adenovirus (Ad5) NKT cell epitope.

| Peptide- | Oxidoreductase | | | | | |
|---|---|---|---|---|---|---|
| Noº | Motif | | Linker | T-Epitope | C-term | SEQ ID NO |
| 37 | — | CPYC | GG | FIGLMYY | | 375 |
| 38 | D | CPYC | GG | FIGLMYY | | 376 |
| 39 | E | CPYC | GG | FIGLMYY | | 377 |
| 40 | S | CPYC | GG | FIGLMYY | | 378 |
| 41 | T | CPYC | GG | FIGLMYY | | 379 |
| 42 | N | CPYC | GG | FIGLMYY | | 380 |
| 43 | Q | CPYC | GG | FIGLMYY | | 381 |
| 44 | C | CPYC | GG | FIGLMYY | | 382 |
| 45 | G | CPYC | GG | FIGLMYY | | 383 |
| 46 | P | CPYC | GG | FIGLMYY | | 384 |
| 47 | A | CPYC | GG | FIGLMYY | | 385 |
| 48 | I | CPYC | GG | FIGLMYY | | 386 |
| 49 | L | CPYC | GG | FIGLMYY | | 387 |
| 50 | M | CPYC | GG | FIGLMYY | | 388 |
| 51 | F | CPYC | GG | FIGLMYY | | 389 |
| 52 | W | CPYC | GG | FIGLMYY | | 390 |
| 53 | Y | CPYC | GG | FIGLMYY | | 391 |
| 54 | V | CPYC | GG | FIGLMYY | | 392 |

TABLE 4
Immunogenic peptides with different oxidoreductase motifs based on
the CPYC motif and a MOG MHC class II T cell epitope.

| Peptide- | Oxidoreductase | | | | | |
|---|---|---|---|---|---|---|
| Noº | Motif | | Linker | T-Epitope | C-term | SEQ ID NO |
| 55 | — | CPYC | GW | YRSPFSRVV | HLYR | 393 |
| 56 | D | CPYC | GW | YRSPFSRVV | HLYR | 394 |
| 57 | E | CPYC | GW | YRSPFSRVV | HLYR | 395 |
| 58 | S | CPYC | GW | YRSPFSRVV | HLYR | 396 |
| 59 | T | CPYC | GW | YRSPFSRVV | HLYR | 397 |
| 60 | N | CPYC | GW | YRSPFSRVV | HLYR | 398 |
| 61 | Q | CPYC | GW | YRSPFSRVV | HLYR | 399 |
| 62 | C | CPYC | GW | YRSPFSRVV | HLYR | 400 |
| 63 | G | CPYC | GW | YRSPFSRVV | HLYR | 401 |
| 64 | P | CPYC | GW | YRSPFSRVV | HLYR | 402 |
| 65 | A | CPYC | GW | YRSPFSRVV | HLYR | 403 |
| 66 | I | CPYC | GW | YRSPFSRVV | HLYR | 404 |
| 67 | L | CPYC | GW | YRSPFSRVV | HLYR | 405 |
| 68 | M | CPYC | GW | YRSPFSRVV | HLYR | 406 |
| 69 | F | CPYC | GW | YRSPFSRVV | HLYR | 407 |
| 70 | W | CPYC | GW | YRSPFSRVV | HLYR | 408 |
| 71 | Y | CPYC | GW | YRSPFSRVV | HLYR | 409 |
| 72 | V | CPYC | GW | YRSPFSRVV | HLYR | 410 |

TABLE 5
Immunogenic peptides with different oxidoreductase motifs based on
the CGC motif and a MOG MHC class II T cell epitope.

| Peptide- | Oxidoreductase | | | | | |
|---|---|---|---|---|---|---|
| Noº | Motif | | Linker | T-Epitope | C-term | SEQ ID NO |
| 73 | — | CGC | GW | YRSPFSRVV | HLYR | 411 |
| 74 | D | CGC | GW | YRSPFSRVV | HLYR | 412 |
| 75 | E | CGC | GW | YRSPFSRVV | HLYR | 413 |
| 76 | S | CGC | GW | YRSPFSRVV | HLYR | 414 |
| 77 | T | CGC | GW | YRSPFSRVV | HLYR | 415 |
| 78 | N | CGC | GW | YRSPFSRVV | HLYR | 416 |
| 79 | Q | CGC | GW | YRSPFSRVV | HLYR | 417 |
| 80 | C | CGC | GW | YRSPFSRVV | HLYR | 418 |
| 81 | G | CGC | GW | YRSPFSRVV | HLYR | 419 |
| 82 | P | CGC | GW | YRSPFSRVV | HLYR | 420 |
| 83 | A | CGC | GW | YRSPFSRVV | HLYR | 421 |
| 84 | I | CGC | GW | YRSPFSRVV | HLYR | 422 |
| 85 | L | CGC | GW | YRSPFSRVV | HLYR | 423 |
| 86 | M | CGC | GW | YRSPFSRVV | HLYR | 424 |
| 87 | F | CGC | GW | YRSPFSRVV | HLYR | 425 |
| 88 | W | CGC | GW | YRSPFSRVV | HLYR | 426 |
| 89 | Y | CGC | GW | YRSPFSRVV | HLYR | 427 |
| 90 | V | CGC | GW | YRSPFSRVV | HLYR | 428 |

TABLE 6
Immunogenic peptides with different oxidoreductase motifs based on the
CPYC motif and an insulin MHC class II T cell epitope.

| Peptide- | Oxidoreductase | | | | | |
|---|---|---|---|---|---|---|
| Noº | Motif | | Linker | T-Epitope | C-term | SEQ ID NO |
| 91 | W | CPYC | SLQP | LALEGSLQK | RG | 429 |
| 92 | P | CPYC | SLQP | LALEGSLQK | RG | 430 |
| 93 | Y | CPYC | SLQP | LALEGSLQK | RG | 431 |
| 94 | F | CPYC | SLQP | LALEGSLQK | RG | 432 |
| 95 | V | CPYC | SLQP | LALEGSLQK | RG | 433 |
| 96 | I | CPYC | SLQP | LALEGSLQK | RG | 434 |
| 97 | M | CPYC | SLQP | LALEGSLQK | RG | 435 |
| 98 | C | CPYC | SLQP | LALEGSLQK | RG | 436 |
| 99 | L | CPYC | SLQP | LALEGSLQK | RG | 437 |
| 100 | G | CPYC | SLQP | LALEGSLQK | RG | 438 |
| 101 | A | CPYC | SLQP | LALEGSLQK | RG | 439 |
| 102 | Q | CPYC | SLQP | LALEGSLQK | RG | 440 |
| 103 | T | CPYC | SLQP | LALEGSLQK | RG | 441 |
| 104 | N | CPYC | SLQP | LALEGSLQK | RG | 442 |
| 105 | S | CPYC | SLQP | LALEGSLQK | RG | 443 |
| 106 | E | CPYC | SLQP | LALEGSLQK | RG | 444 |
| 107 | D | CPYC | SLQP | LALEGSLQK | RG | 445 |
| 108 | H | CPYC | SLQP | LALEGSLQK | RG | 446 |

### Example 2: Methodology to Assess Reducing Activity of Peptides

The reductase activity of the peptides is determined using a fluorescent assay described in Tomazzoli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again. All the tests with these peptides were performed in duplicates, and each test was conducted two times. Control experiments are performed with dithiothreitol (100% reducing activity) and water (0% reducing activity).

The peptides of the invention are tested for their reducing activity.

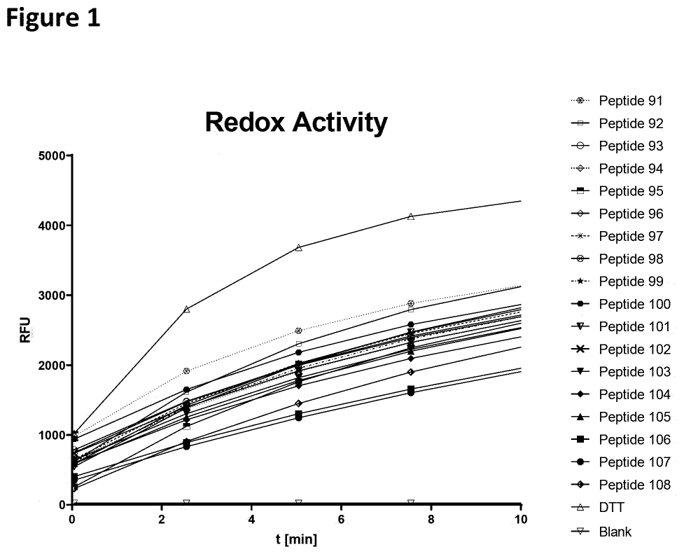

FIG. 1 represents kinetics of reducing activity of peptides 91 to 108 (see table 6). All the peptides tested exhibited a higher reducing activity as compared to the prior art peptide with the motif HCPYC (peptide 108, SEQ ID NO: 447), except peptides with an oxidoreductase motif comprising a negatively charged amino acid E or D (peptides 106 and 107). Immunogenic peptides with bulky hydrophobic amino acids in front of the oxidoreductase motif, such as W, P or G, displayed the highest reducing activity.

### Example 3: Interferon Gamma Release by Cytolytic CD4+ T Cell Lines

Interferon gamma is an important marker to characterise cytolytic CD4+ T cells.

A specific CD4+ T cell line can be obtained by priming and stimulating naïve CD4+ T cells from a T1D patient (T1D07) with an immunogenic peptide. After multiple (e.g.) 12 stimulations, cells can be co-cultured with autologous LCL B cells loaded (2 μM) with said immunogenic peptide. After 24 hours, supernatants are collected and IFN-gamma is measured by multiplex assay.

### Example 4: FasL Release by Cytolytic CD4+ T Cell Lines

The T cell line originally generated with the immunogenic peptide as explained in Example 3 above can be divided and stimulated with said immunogenic peptide over 4 successive in vitro stimulations using an autologous LCL B cell line as APC. At day 11 of every stimulation (total of 4), cells are tested for FasL after restimulation with their corresponding peptide presented by autologous B cells. Supernatants are collected after 24h (stimulation 1 and 2) or 72h (stimulation 3 and 4) of co-culture.

1. An immunogenic peptide, said immunogenic peptide comprising:
a) an oxidoreductase motif,
b) an MHC class II T-cell epitope of an antigenic protein, and
c) a linker between a) and b) of between 0 and 7 amino acids,
wherein said oxidoreductase motif has the following sequence:
Z(B)ₙ[CST]XₘC- or Z(B)ₙCXₘ[CST]-;
wherein Z is any amino acid or non-natural amino acid, excluding basic amino acids comprising R (Arginine), K (Lysine) and H (Histidine), and excluding amino acids D (Aspartate), W (Tryptophane), E (Glutamate), and A (Alanine);
wherein (B) is any amino acid;
wherein n is an integer of 0 to 2;
wherein Cis Cysteine, S is Serine and T is Threonine;
wherein X is any amino acid;
wherein m is 2, 0, 1, or 3; and
wherein the hyphen (-) in said oxidoreductase motif indicates the point of attachment of the oxidoreductase motif to the N-terminal end of the linker (c) or the epitope (b), or to the C-terminal end of the linker (c) or the epitope (b).

2. The immunogenic peptide according to claim 1, wherein Z is selected from the group consisting of: G (Glycine), S, T, C, V (Valine), L (Leucine), I (Isoleucine), M (Methionine), P (Proline), F (Phenylalanine), Y (Tyrosine), N (Asparagine), and Q (Glutamine).

3. The immunogenic peptide according to claim 1, wherein Z is selected from the group consisting of amino acids: G (Glycine), and P (Proline).

4. The immunogenic peptide according to claim 1, wherein m is 2.

5. The immunogenic peptide according to claim 1, wherein X is any amino acid excluding C, S, and T.

6. The immunogenic peptide according to claim 1, wherein one or more X is a basic amino acid selected from the group consisting of R, K and H.

7. (canceled)

8. The immunogenic peptide according to claim 1, wherein said epitope has a length of between 9 and 30 amino acids.

9. The immunogenic peptide according to claim 1, having a length of between 12 and 50 amino acids.

10. The immunogenic peptide according to claim 1, wherein said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen.

11. The immunogenic peptide according to claim 1, wherein the linker is of between 0 and 4 amino acids.

12. The immunogenic peptide according to claim 1, wherein said oxidoreductase motif does not naturally occur within a region of 11 amino acids N-terminally or C-terminally of the MHC class II T-cell epitope in said antigenic protein.

13. The immunogenic peptide according to claim 1, wherein the MHC class II T-cell epitope does not naturally comprise said oxidoreductase motif.

14. The immunogenic peptide according to claim 1, wherein at least one X in the oxidoreductase motif is P (proline) or Y (Tyrosine).

15-17. (canceled)

18. A polynucleotide encoding the immunogenic peptide according to claim 1.

19-22. (canceled)

23. A method for preparing an immunogenic peptide according to claim 1, comprising the steps of:
(a) providing a peptide sequence consisting of an MHC class II T-cell epitope of said antigenic protein, and
(b) linking to said peptide sequence the oxidoreductase motif, such that said motif and said epitope are either adjacent to each other or separated by a linker of at most 7 amino acids.

24. A method for obtaining a population of antigen-specific cytolytic CD4+ T cells, against APC presenting said antigen, the method comprising the steps of:
providing peripheral blood cells;
contacting said cells with an immunogenic peptide according to claim 1, or with a polynucleotide encoding such an immunogenic peptide; and
expanding said cells in the presence of IL-2.

25. A method for obtaining a population of antigen-specific cytolytic CD4+ T cells, against APC presenting said antigen, the method comprising the steps of:
providing an immunogenic peptide according to claim 1 or with a polynucleotide encoding such an immunogenic peptide;
administering said peptide or polynucleotide to a subject; and
obtaining said population of antigen-specific cytolytic CD4+ T cells from said subject.

26. A population of antigen-specific cytolytic CD4+ T cells obtained by the method of claim 24.

27. A method of treating an autoimmune disease, an infection with an intracellular pathogen, a tumor, an allograft rejection, or an immune response to a soluble allofactors, to an allergen exposure or to a viral vector used for gene therapy or gene vaccination in an individual, comprising administering the immunogenic peptide according to claim 1, or a polynucleotide encoding the immunogenic peptide according to claim 1 to said individual.

28. A method of treating an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of:
providing peripheral blood cells of said individual,
contacting said cells with an immunogenic peptide according to any of claim 1 or with a polynucleotide encoding the immunogenic peptide of claim 1,
expanding said cells, and
administering said expanded cells to said individual.
